(19) United States
(12) Patent Application Publication
(10) Pub. No.: US 2026/0035643 A1
(43) Pub. Date: Feb. 5, 2026

(54) PHYSIOLOGIC AIRWAY MUCOSA AND PARENCHYMA BIOREACTOR AND EX VIVO WHOLE LUNG MODEL OF CYSTIC FIBROSIS FOR DISEASE MODELING AND THERAPEUTIC SCREENING

(71) Applicants: The Trustees of Columbia University in the City of New York, New York, NY (US); The Trustees of The Stevens Institute of Technology, Hoboken, NJ (US); Vanderbilt University, Nashville, TN (US)

(72) Inventors: Gordana VUNJAK-NOVAKOVIC, New York, NY (US); Meghan R. PINEZICH, New York, NY (US); Maria R. HUDOCK, New York, NY (US); Jinho KIM, Jersey City, NJ (US); Keith YEAGER, Springfield, NJ (US); Panpan CHEN, New York, NY (US); Julie VAN HASSEL, New York, NY (US); Matthew BACCHETTA, Tenafly, NJ (US)

(21) Appl. No.: 19/355,648

(22) Filed: Oct. 10, 2025

Related U.S. Application Data
(63) Continuation of application No. PCT/US2024/024088, filed on Apr. 11, 2024.
(60) Provisional application No. 63/495,977, filed on Apr. 13, 2023.
(60) Provisional application No. 63/507,001, filed on Jun. 8, 2023.

(51) Int. Cl. C12M 3/00; C12M 1/00; C12M 1/12; C12M 1/32; C12M 1/34

(52) U.S. Cl. CPC C12M 21/08; C12M 23/12; C12M 23/22; C12M 23/38; C12M 23/44; C12M 23/48; C12M 23/58; C12M 29/10; C12M 29/18; C12M 37/04; C12M 41/14; C12M 41/34; C12M 41/40

(57) ABSTRACT

Modular mucosal tissue bioreactors combined with bioartificial mucus provide a high-fidelity, medium-throughput platform for evaluating CF treatments and modeling host-pathogen biology, a stepping stone towards better options for HEMT-nonresponsive patients. Whole lung ex vivo Cystic Fibrosis model system allows for investigation of candidate CF therapies.

### CROSS-REFERENCE TO RELATED APPLICATIONS

This application is a continuation of PCT International Application No. PCT/US2024/024088, filed Apr. 11, 2024, which claims benefit of U.S. Provisional Application No. 63/495,977, filed Apr. 13, 2023, and of U.S. Provisional Application No. 63/507,001, filed Jun. 8, 2023, the contents of each of which are hereby incorporated by reference.

### GOVERNMENT SUPPORT CLAUSE

This invention was made with government support under EB027062, HL134760 and HL120046 awarded by the National Institutes of Health. The government has certain rights in the invention.

### BACKGROUND

Cystic fibrosis (CF) is a monogenic disease on the frontier of many therapeutic strategies. CF is a rare disease that causes the lungs to generate large amounts of concentrated mucus. CF gene mutations are carried by 1 in 30 people, and CF affects approximately 40,000 Americans today. Highly effective modulator therapy (HEMT) has improved the quality of life for a majority of CF patients, but HEMT is not curative—and for some patients, HEMT does not work at all. There is still a need for more effective antibiotics, mucolytics, and disease-modifying drugs that can help all patients, regardless of their mutations. All of these therapies can be enhanced by better drug delivery methods.

CF lung disease is difficult to model. Animal models fail to recapitulate all aspects of the pulmonary disease, with the B-ENaC mouse coming the closest to capturing the phenotype, but without the relevant genotype. Cell culture models lose some systemic effects (immune system), structure, and significant inflammatory mucus accumulation.

A lack of high-fidelity animal models or testing platforms hinders drug delivery studies. There is a need for a novel preclinical model of the diseased airways of CF.

Despite the emergence of highly effective modulator therapies (HEMT), many patients with Cystic Fibrosis (CF) still need better symptomatic, disease-modifying, and infection-targeted treatment. Obstacles to therapeutic development include limited fidelity or intensive care requirements of animal models, and the difficulty of recapitulating the thick airway mucus that protects pathogens and hinders drug delivery.

A high throughput model that recapitulates the extracellular environment is needed for successful translation of therapies that cover all patients.

In addition, despite decades of research, successful translation of CF gene therapy remains hampered by the lack of high-fidelity pre-clinical models for therapeutic screening and optimization. Existing culture systems are overly simplistic and do not recapitulate the full range of stimuli encountered in the CF lungs, and animal models do not develop a representative CF phenotype, or develop illness so severe that animal care is prohibitively intensive. As a result, there is an unmet need for improved pre-clinical models of CF to screen gene therapy candidates and enable therapeutic translation.

### SUMMARY

To address these obstacles, a high-fidelity in vitro CF model is provided comprising a modular mucosal tissue bioreactor and bioartificial CF mucus for evaluating CF treatments and host-pathogen interactions. More broadly, a high-fidelity in vitro airway tissue model is provided comprising a modular mucosal tissue bioreactor and, optionally a bioartificial mucus for us in, for example, evaluating treatments and host-pathogen interactions.

One aspect provides an air-liquid interface bioreactor with integrated perfusion and ventilation that supports a thin (<600 μm) slice of mucosa from porcine or human airways.

Embodiments include the following, alone or in any combination.

The bioreactor may comprise a base comprising one or more wells for holding culture medium; a platform for supporting the mucosa in contact with the culture medium in the one or more wells; a fluid circulating loop for circulating culture medium through the one or more wells; and an air circulation loop for circulating air over a top surface of the mucosa.

Each well may comprise an entry side and a removal side separated by a half wall; wherein the entry side comprises an opening proximate a floor of the entry side in fluid communication with tubing of the fluid circulation loop; and the removal side comprises a downward-facing tube that opens just above a level of a floor of the removal side in fluid communication with tubing of the fluid circulation loop.

The bioreactor may comprise a chair-like tissue holder comprising a polymeric frame with a permeable mesh seat on which tissue slices are placed.

The tissue slices may be attached to the seat with collagen hydrogels or other adhesive measures.

The legs of the chair-like tissue holder may plug into complementarily designed holes in the floor of the reactor to stabilize the holder's position.

The seat of the chair may be disposed near the height of the culture medium in the one or more wells in such a way that the tissue is maintained in an air-liquid interface wherein a top of tissue is exposed to air and a bottom of the tissue is submerged in the culture medium.

The bioreactor may be configured to be compatible with both epifluorescence and light sheet microscopy.

The bioreactor may comprise a lid comprising a central opening just above the location of the tissue in its chair-like holder, wherein the opening is optionally 1) left open to the air, for easy loading and removal of the entire tissue and/or chair-like tissue holder, 2) sealed with a coverslip and adhesive, or 3) sealed with a custom insert that holds a recessed coverslip deeper in the chamber for the purpose of bringing imaging equipment in closer proximity to the surface of the tissue.

The bioreactor may comprise one or more windows in a side wall of the reactor that is positioned such that a side profile of the tissue, tissue holder, culture medium below the tissue and mucus/air above the tissue are observable.

The bioreactor may comprise a peristaltic pump, and a heating system using a heating pad with a PID controller and digital readout.

A plurality of bioreactors of claim 1 maintained in parallel in a standard incubator. Modularity permits maintenance of many bioreactor units in parallel in a standard incubator. Each unit is compatible with both epifluorescence and light sheet microscopy.

We also generated a bioartificial mucus benchmarked to patient sputum in macromolecular composition, rheology, dextran permeability, and microrheology via particle tracking. Embodiments of the bioartificial mucus comprise DNA, phospholipids, and mucin to create a solution that mimics CF mucus.

A mucosal tissue bioreactor comprising:
a base comprising one or more wells that support a culture medium;
one or more platforms configured to support a portion of an airway mucosa in at least partial contact with the culture medium in the one or more wells;
a fluid circulation loop that circulates the culture medium through the one or more wells; and
an air circulation loop that circulates air over a top surface of the portion of the airway mucosa.

An air-liquid interface bioreactor with integrated perfusion and ventilation that supports a slice of mammalian airway mucosa in culture comprising:
a base comprising one or more wells for holding a culture medium;
one or more platforms for supporting the mucosa in contact with the culture medium in the one or more wells;
a fluid circulation loop for circulating culture medium through the one or more wells; and
an air circulation loop for circulating air over a top surface of the mucosa.

A plurality of bioreactors as described herein, each comprising a portion of an airway mucosa slice.

A bioartificial mucus benchmarked to a patient sputum in macromolecular composition, rheology, dextran permeability, and microrheology via particle tracking. In embodiments the bioartificial mucus comprises DNA, phospholipids, and mucin to create a solution that mimics CF mucus.

A whole lung model of cystic fibrosis comprising
an ex vivo lung supported with a cross-circulation apparatus,
bioartificial mucus present in the ex vivo lung airways which mimics mucus of cystic fibrosis, integrated real-time imaging and/or analytic tools for assessment of lung function and therapeutic uptake and efficacy.

A method of ex vivo screening of a candidate therapeutic for cystic fibrosis comprising administering the candidate therapeutic to the lung in the system described herein and determining one or more of candidate therapeutic uptake, distribution, and efficacy in real-time.

A method of ex vivo screening of a candidate therapeutic for cystic fibrosis comprising administering the candidate therapeutic to the lung in the system described herein and determining a status of the lung before and after administration of the candidate therapeutic for cystic fibrosis so as to determine if the candidate therapeutic improves one or more symptoms of cystic fibrosis.

### BRIEF DESCRIPTION OF FIGURES

FIG. 1: Design considerations for aspects of a physiologic airway mucosa and parenchyma bioreactor.

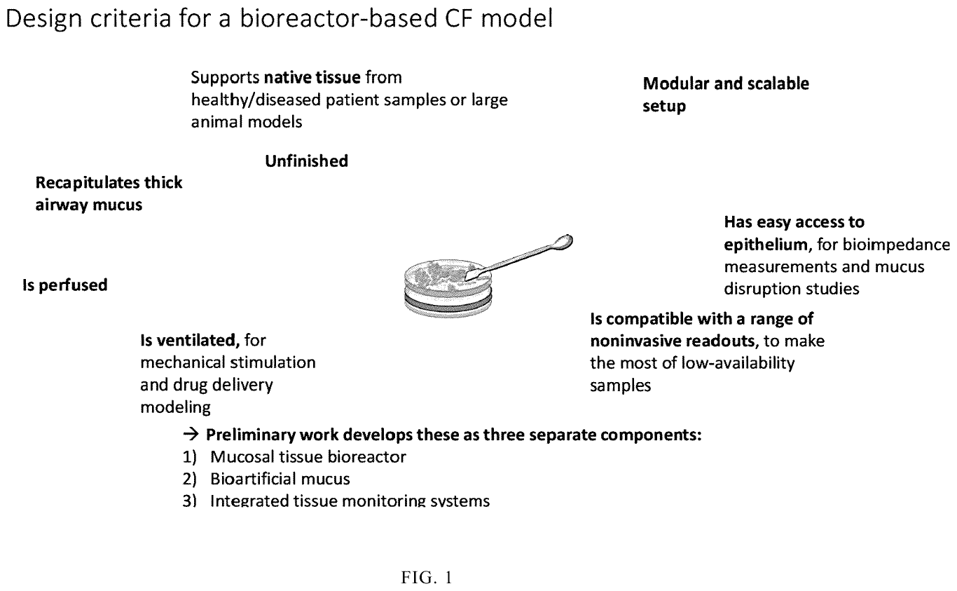

FIG. 2: Exemplary mucosal tissue bioreactor components including an example of a chair-like tissue holder.

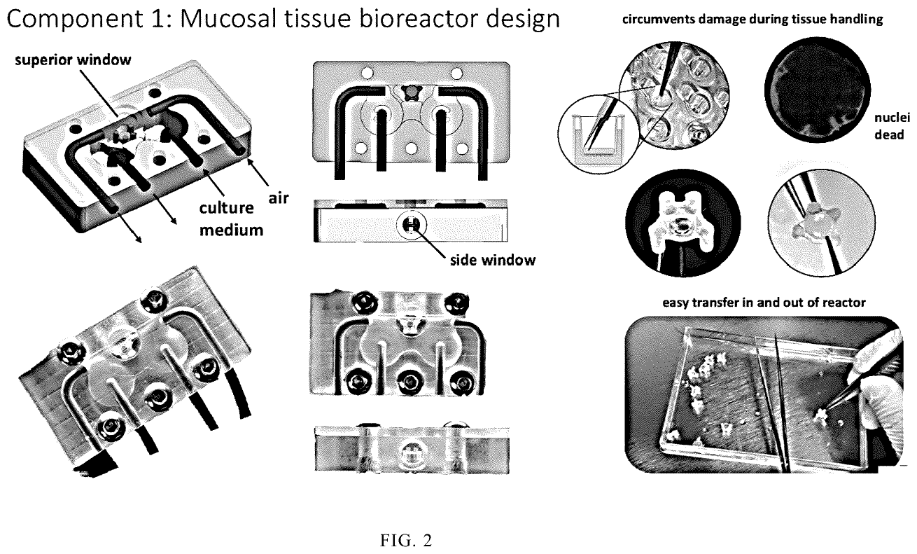

FIG. 3: Hoeschst/propidium iodide results showing good integrity of mucosa and submucosal layers maintained in bioreactor after dissection at one week.

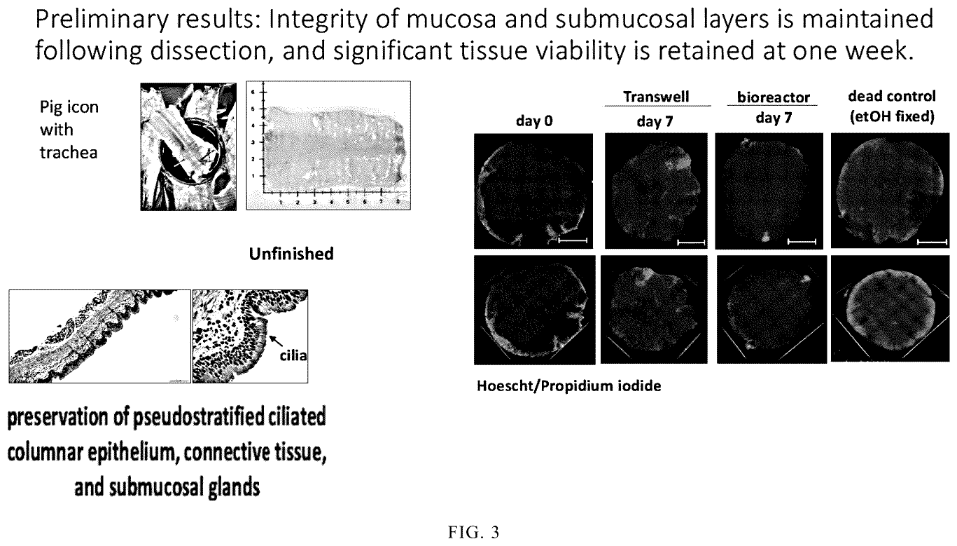

FIG. 4: Design considerations for aspects of an artificial mucus.

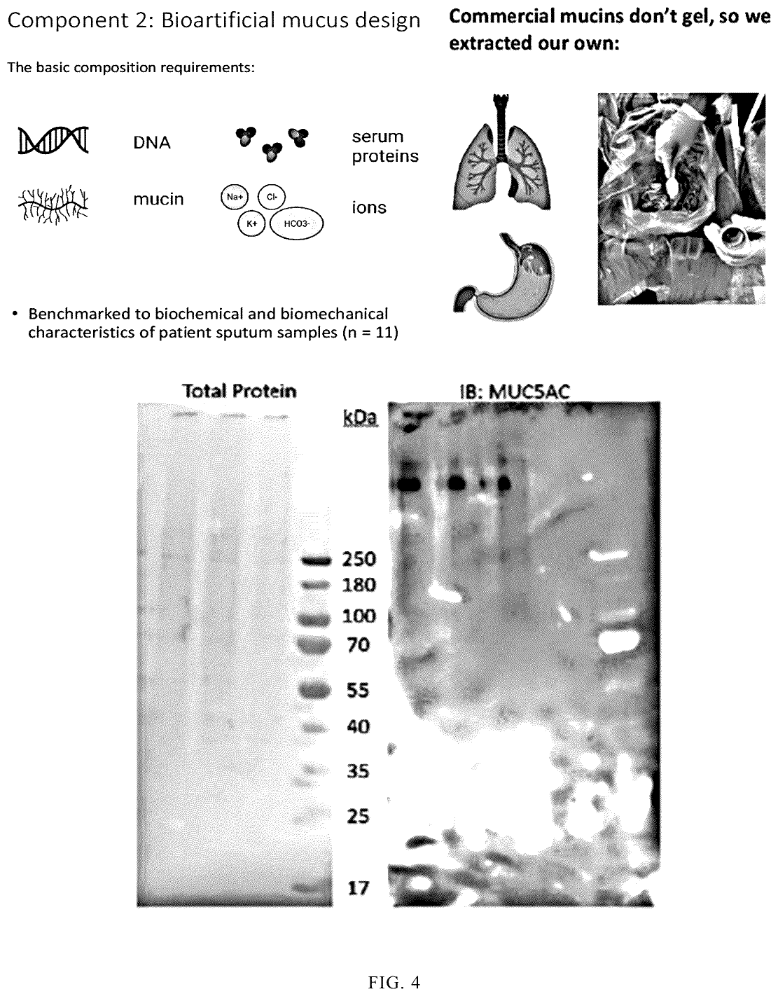

FIG. 5: Macroscopic and microscopic viscosity of bioartificial mucus as determined by macrorheology and microrheology.

FIG. 6: Macroscopic and microscopic viscosity of bioartificial mucus as determined by macrorheology and microrheology.

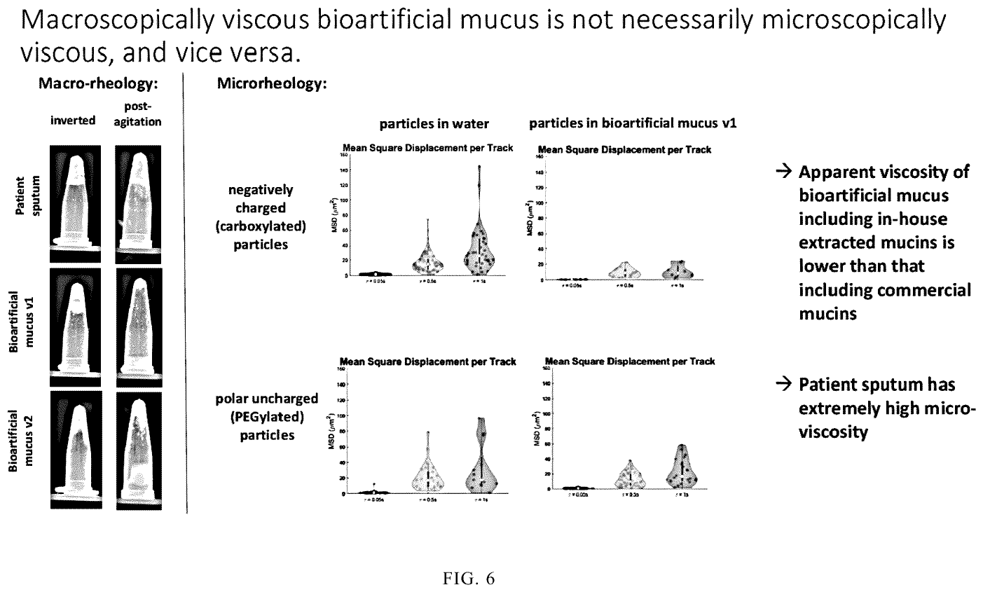

FIG. 7: Bioreactor design interaction with non-invasive sensing and imaging technologies.

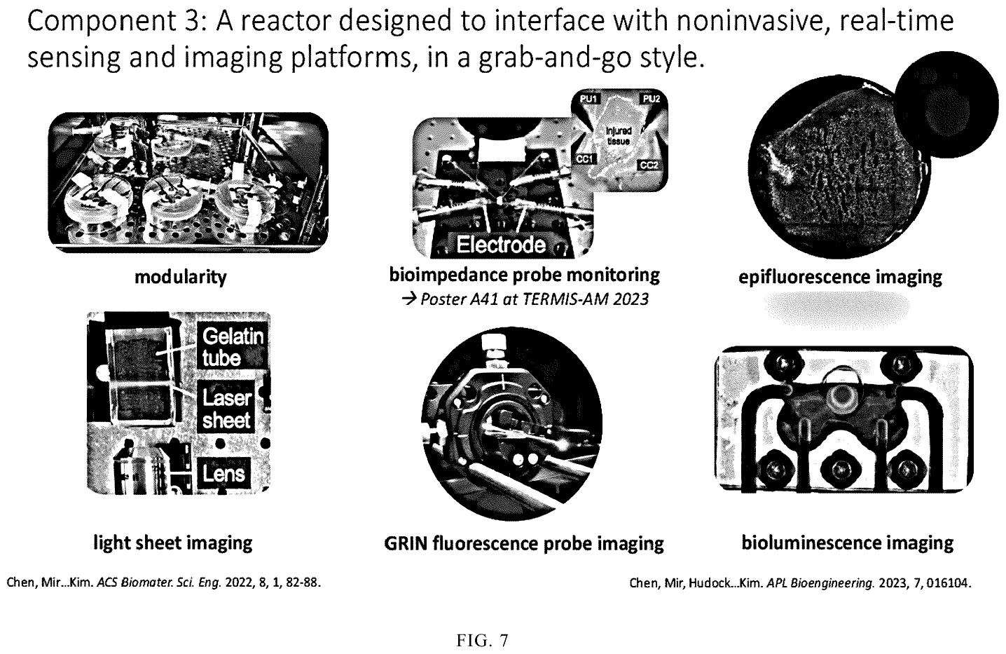

FIG. 8: Fluorescent particle tracing results showing ciliary function in dissected mucosal airway slices maintained at one week after transfer to bioreactor.

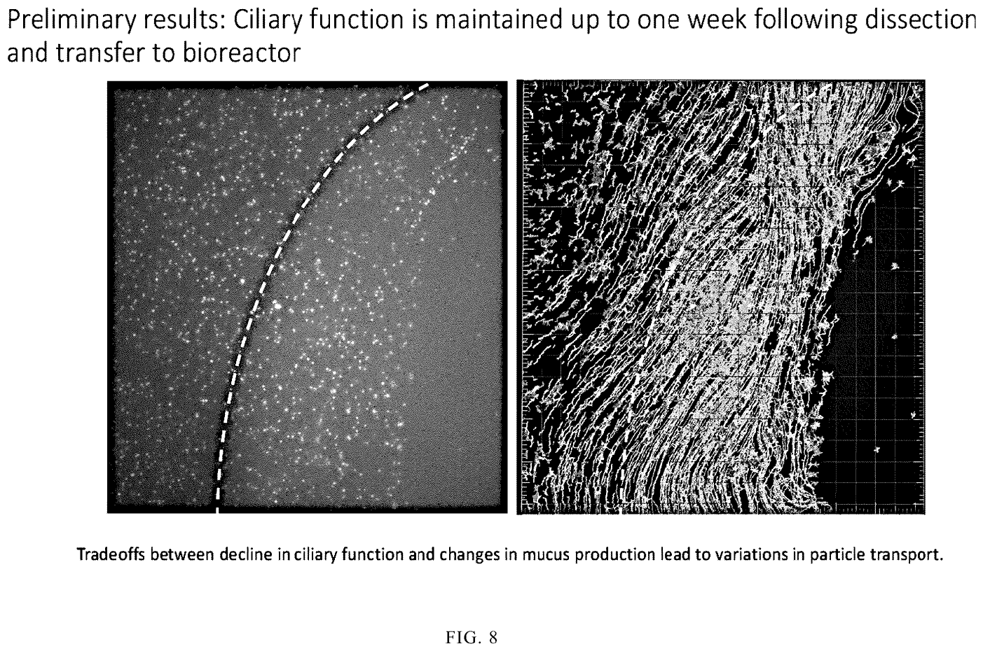

FIG. 9: Other embodiments of physiologic airway mucosa and parenchyma tissue bioreactor design versions: incorporated higher tissue capacity and a lid sealing mechanism, in which, e.g., metal pins, are used to fasten on the lid over an O-ring and create an air-tight seal.

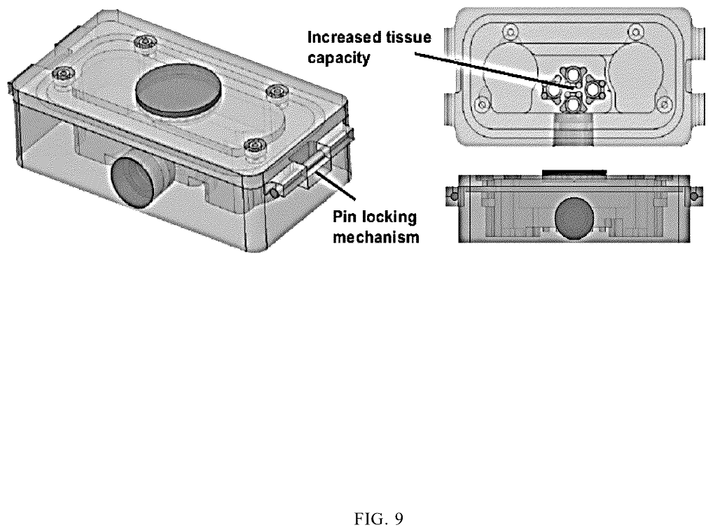

FIG. 10: Other embodiments of physiologic airway mucosa and parenchyma tissue bioreactor design: a living hinge mechanism fastens in a removable imaging window (one such window design shown, but many other variations are possible) that is easily actuated by the user to swap out different windows for different imaging needs.

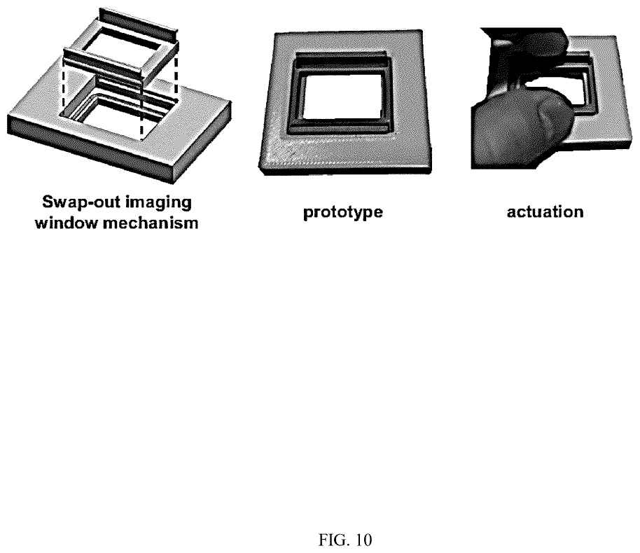

FIG. 11: illustrates the overall approach according to the disclosed CF subject matter.

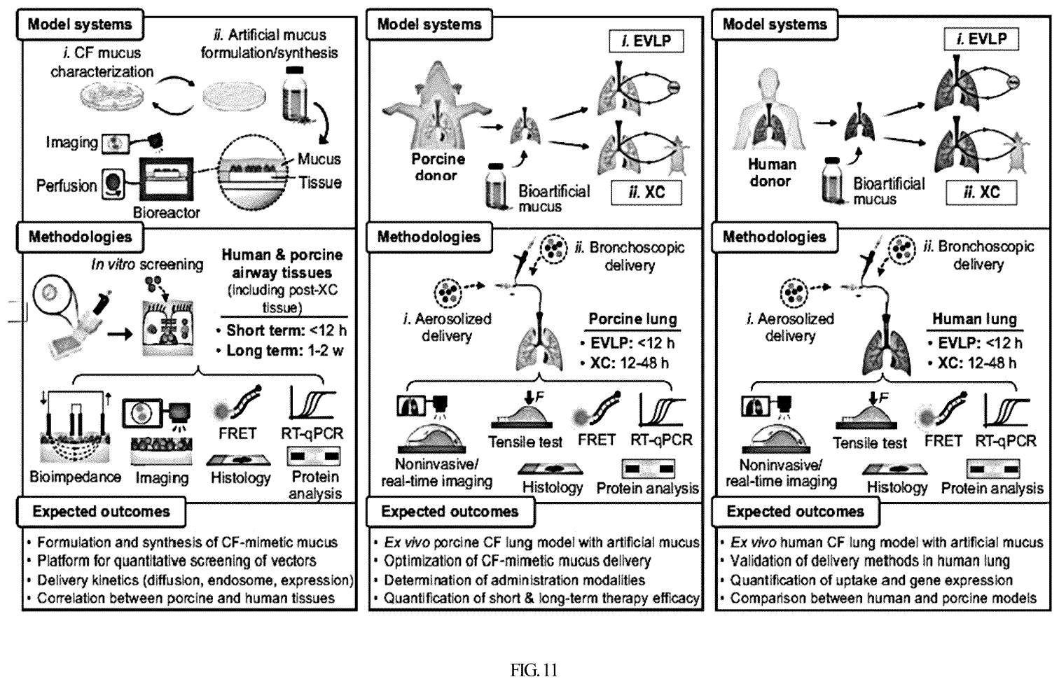

FIGS. 12A-12B: are representative images of carboxylate-modified particles (FIG. 12A) and PEGylated particles (FIG. 12B) diffusing in the same artificial mucus formulation. Note apparent large (˜20 μm) aggregations (not moving in original video) visible only in (FIG. 12A).

FIGS. 13A-13E: illustrate in vitro-cultured airway tissues: (FIG. 13A) (i) Photograph and (ii) schematic of rat trachea bioreactor. (FIG. 13B) (i) A GRIN imaging probe integrated into (ii) the bioreactor. (iii) Fluorescent images of native epithelium before (green) and after (black) de-epithelialization (De-Epith) and cells (red) seeded onto airway lumen. (FIG. 13C) Immunostaining and SEM images of native and de-epithelialized rat tracheas. Cilia and tight junctions indicated by acetylated alpha tubulin (a-Tub, green) and Zonula Occludens-1 (ZO-1, red). (FIG. 13D) Engraftment of seeded cells on the de-epithelialized airway lumen quantified by cell morphology. (FIG. 13E) Prototype mucosal tissue bioreactor (i) schematic and (ii) photograph, with (iii) basic ventilation and perfusion setup (parts 1A-F listed above 13E (iii) in the figure).

FIGS. 14A-14C: illustrate in situ quantification of the mucociliary flow: (FIG. 14A) Monitoring the mucociliary flow by tracking microparticles (MPs) along the airway lumen. Mucociliary flow analysis across (FIG. 14B) native and (FIG. 14C) injured tracheal epithelium. (i) Fluorescent images of rat trachea stained with wheat germ agglutinin (WGA) and Evans blue (EB) to label ciliated cells and injured tissue, respectively. (iii) Trajectories of MPs indicating mucociliary flow.

FIGS. 15A-15B: Particle visualization via laser light sheet microscopy (LSM): (FIG. 15A) A LSM system constructed for visualizing particle or cell distribution. (FIG. 15B) Imaging 10-μm microparticles (green) deposited onto the inner surface of a transparent hydrogel tube.

FIGS. 16A-16E: illustrate measurement and simulation of bioimpedance of airway tissue: (FIG. 16A) Bioimpedance measurement setup created for noninvasive tissue analysis. (FIG. 16B) Microscopic photography of rat trachea lumen with localized injury. (FIG. 16C) Schematic of the cross-section view of the tissue being measured for the bioimpedance. (FIG. 16D) (i) An equivalent electrical circuit and (ii) measured complex impedance of native and injured airway tissue. (FIG. 16E) Computer simulated distribution of electric field across the airway tissue surface near injury site.

FIGS. 17A-17C: illustrate modulation of epithelial permeability: (FIG. 17A) Fluorescent images of (i) rat trachea treated with EDTA and (ii) native tissue (control). (FIG. 17B) Cross-section view of the EDTA-treated tissue acquired via confocal microscopy. (FIG. 17C) Bioimpedance measured from native and EDTA-treated airways.

FIGS. 18A-18C: illustrate enrichment of target-binding nucleic acids: (FIG. 18A) Schematic and (FIG. 18B) a binding model for target (T)-binding sensor(S) attached to anchor strand (FIG. 18A). (FIG. 18C) Gel image of released sensors.

FIGS. 19A-19C: illustrate CF-mimetic in vitro airway tissue model: Schematic of (FIG. 19A) the CF-airway model platform, (FIG. 19B) transport of LNPs across bioartificial mucus and into the epithelium, and (FIG. 19C) gene release monitoring via bioimpedance measurement. Vent: ventilation, Perfu: perfusion, CM: culture medium, Epi: epithelium.

FIG. 20: illustrates optical monitoring of release of gene or mRNA: Nanoparticles can be engineered to emit fluorescent light signals upon release of gene or mRNA from the nanoparticles. F: fluorophore, Q: quencher.

FIG. 21: illustrates nanoparticle delivery to airway basal stem cells (BSCs): To promote the particle delivery to BSCs, the mucociliary barrier and tight/adherens junctions (TAJs) will be disrupted temporarily via mechano-chemical treatment.

FIGS. 22A-22C: illustrate custom-built EVLP platform. (FIG. 22A) Photograph of isolated EVLP setup with online readout capabilities for monitoring lung function. (FIG. 22B) EVLP circuit with integrated in-line circuit components. (FIG. 22C) Representative imaging capabilities including gross, radiographic, and thermographic images.

FIGS. 23A-23B: Cross-circulation platform. (FIG. 23A) Cross-circulation schematic: swine recipient is cannulated through internal jugular (IJ) vein and donor lungs are perfused with recipient whole blood. H, heat exchanger; Q, flow probe; P, pressure probe; T, temperature probe; B, blood sampling port; A, airflow probe; PA, pulmonary artery; PV, pulmonary vein. (FIG. 23B) Hematoxylin & cosin (H&E) stain of pulmonary parenchyma, airways, and vasculature at Day 0 and Day 4.

FIGS. 24A-24F: illustrate imaging modalities. (FIG. 24A) Schematic and (FIG. 24B) photograph of the lung bioreactor coupled with (FIG. 24C) bioluminescence imaging (BLI), (FIG. 24D) thermal imaging, (FIG. 24E) transpleural imaging, and (FIG. 24F) X-ray imaging devices.

FIGS. 25A-25E: illustrate liquid film generation by plug instillation: (FIG. 25A) Gelatin-based tubular channel. (FIG. 25B) Liquid film generation by liquid instillation. (FIG. 25C) Experimental setup. (FIG. 25D) Plug travel distance (S) against plug volume (V₀). (FIG. 25E) Film thickness (h/r) against capillary number of the plug (Ca).

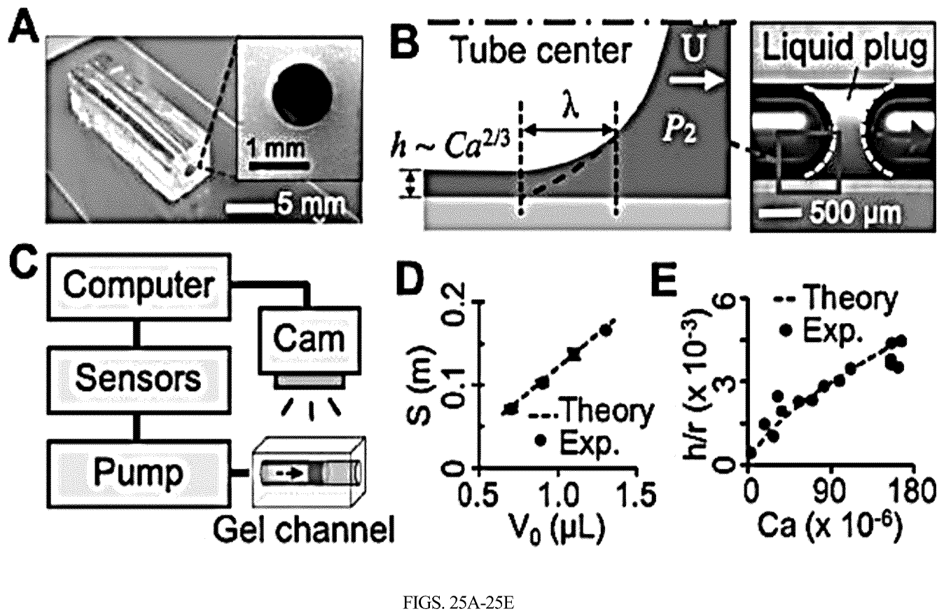

FIGS. 26A-26D: illustrate liquid film generation in the rat lung. (FIG. 26A) Experimental setup with an NIR imaging system. (FIG. 26B) Rat lungs loaded with ICG solution. (FIG. 26C) Flow conditions adjusted via a ventilator for plug instillation. (FIG. 26D) Liquid film deposited in selected anatomical regions.

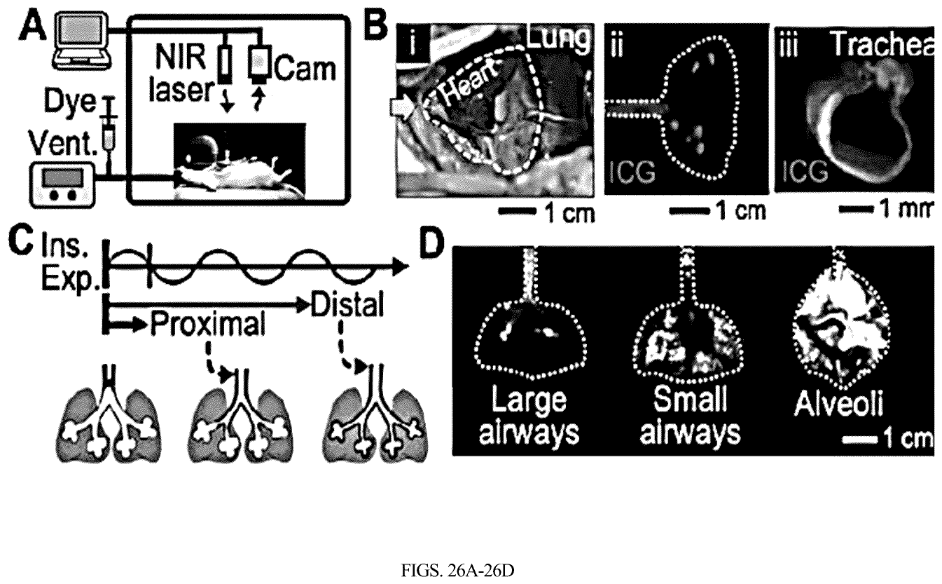

FIGS. 27A-27B: illustrate locally targeted liquid delivery. Liquid film delivery into (FIG. 27A) upper (G₀-G₅) and (FIG. 27B) small airways (G₀-G₁₅).

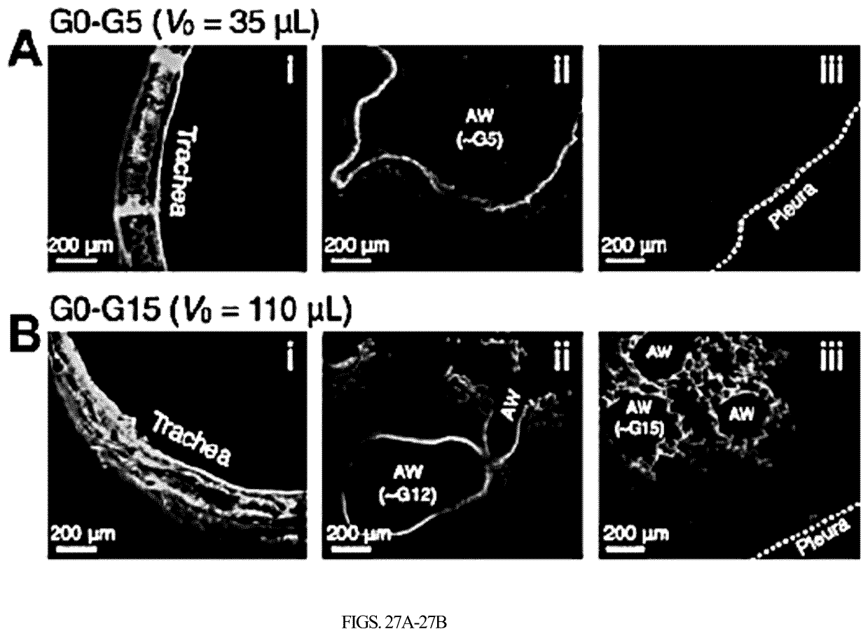

FIGS. 28A-28E: illustrate mechanical property measurement of ex vivo rat lungs. (FIG. 28A) (i) Photograph and (ii) schematic of setup for measuring lung tissue stiffness. (FIG. 28B) Lung tissue subjected to vacuum pressure (−2 kPa) via a capillary tube. (FIG. 28C) Elongation length (LE) measured with varied vacuum pressure. (FIG. 28D) Maximum elongation length (LE). (FIG. 28E) Elastic moduli of tissue with different P_{Alv.}**p<0.05

FIG. 29: illustrates in situ imaging of delivered nanotherapeutics. Delivery of labeled nanoparticles and monitoring the lung with transpleural, near-infrared, and x-ray imaging modalities.

FIGS. 30A-30C: illustrate human lungs supported with xenogeneic cross-circulation for 24 hours: (FIG. 30A) Gross appearance of the lung and (FIG. 30B) scanning electron microscopy for visualization of alveolar structure. (FIG. 30C) Maintenance of pulmonary cilia after 24 h of cross circulation.

FIGS. 31A-31B: illustrate immunogenic interactions on xenogeneic cross-circulation (FIG. 31A) Immunohistochemical staining of swine and human CD45+ leukocyte deposition in human lungs on cross-circulation. (FIG. 31B) Quantification of swine and human CD45+ leukocytes.

FIG. 32: Cross-circulation support results-multiday maintenance allows longitudinal analysis of functional changes.

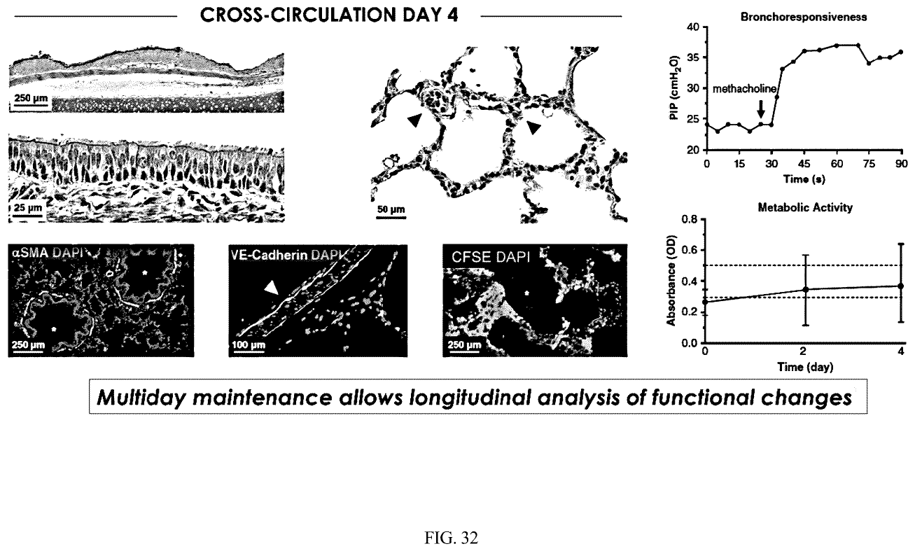

FIG. 33: Cross-circulation support for ex vivo CF lung or normal lung with mucus added.

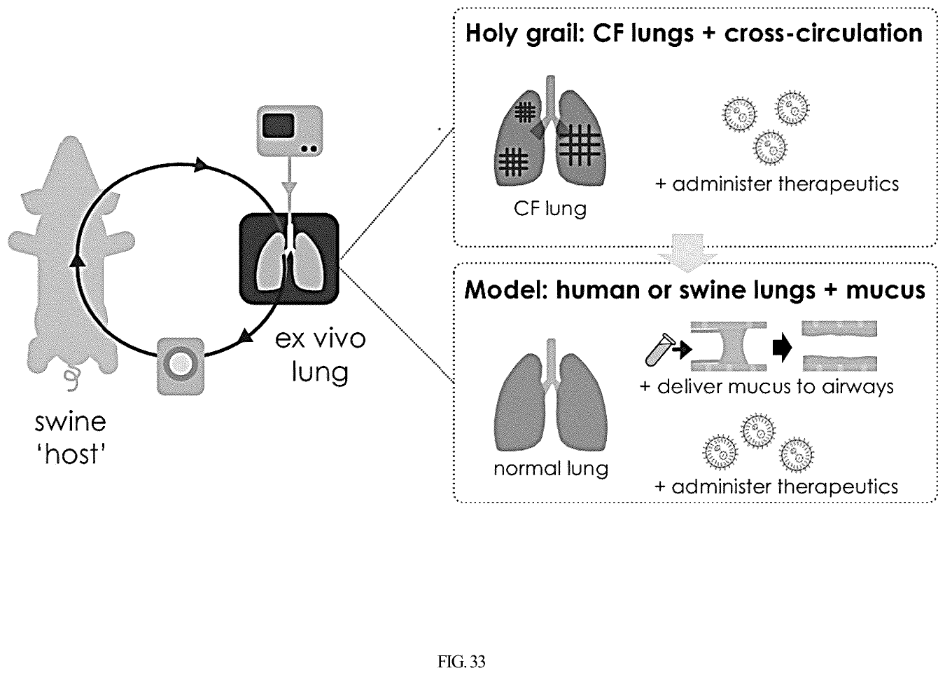

FIG. 34: Delivery of mucus to ex vivo lung.

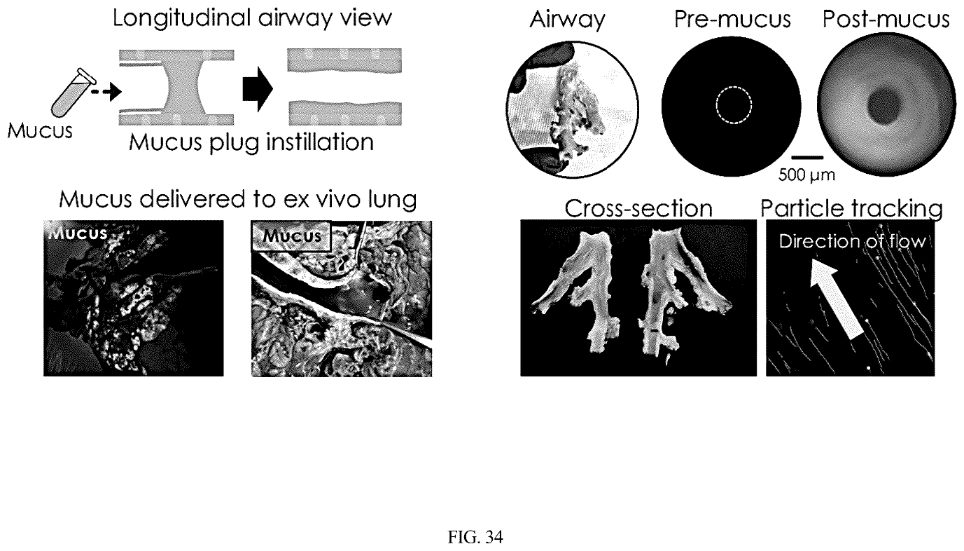

FIG. 35: Cross-circulation support for bronchoscopic, delivery, bronchial blocker and nebulization.

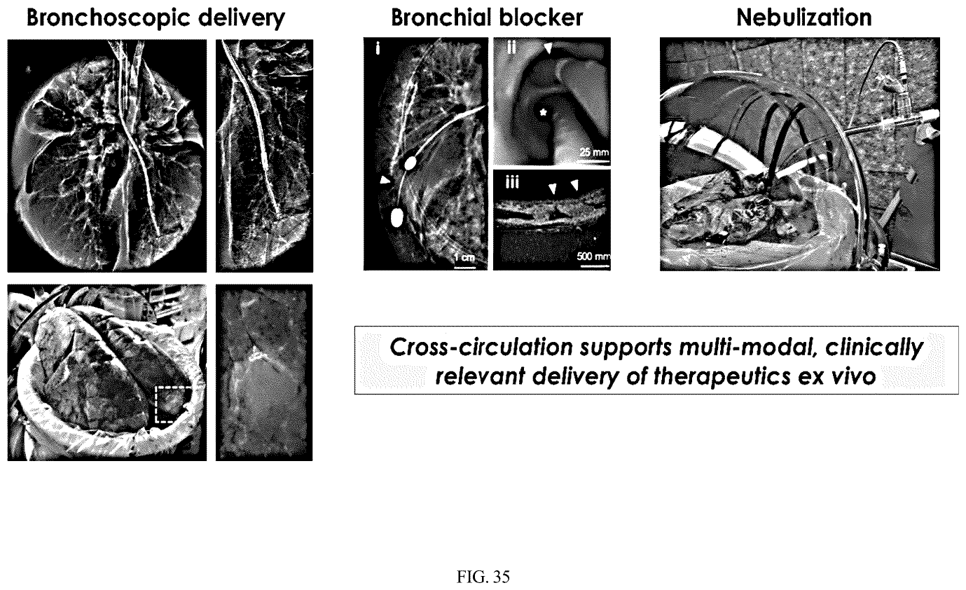

FIG. 36: Transpleural fluorescent scope, cell delivery and microparticle delivery.

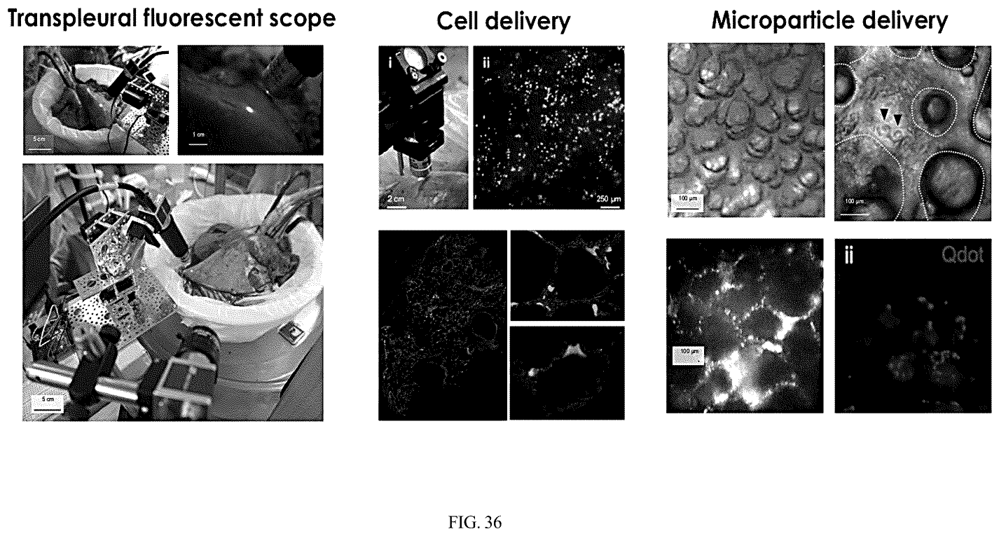

FIG. 37: Whole lung bioluminescence platform and delivery and visualization of bioluminescent cargo.

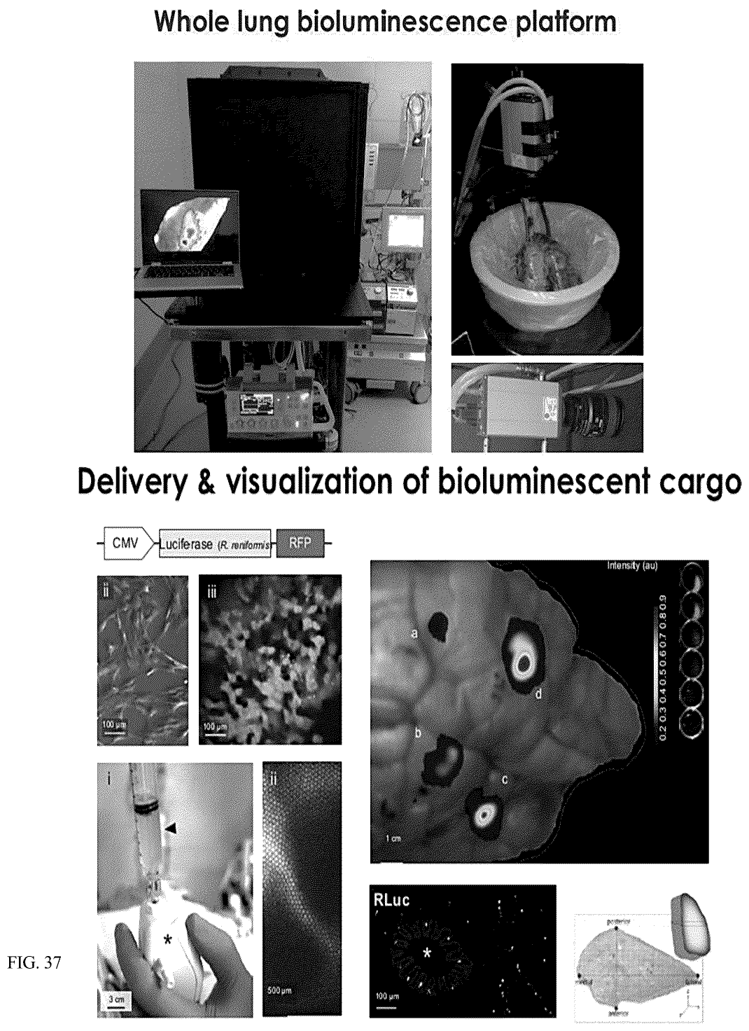

FIG. 38: Procedure room setup for ex vivo lung model maintenance and real-time analysis. (A) Ex vivo lungs are maintained on cross-circulation via perfusion with swine host whole blood and mechanical ventilation with integrated real-time analysis. (B) Advanced biosensing and imaging performed during cross-circulation includes airway bioimpedance, fluorescent bronchoscopy, and pressure-volume (PV) loop measurements.

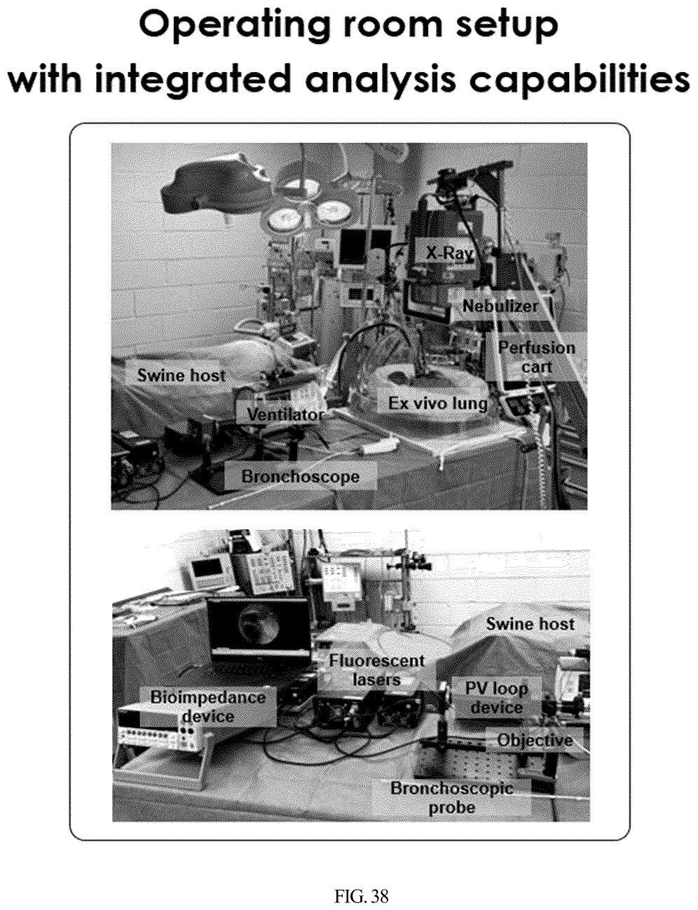

FIG. 39: Apparatus and method or mucus delivery to ex vivo lungs. (A) A custom-built, adjustable lung tilt table was constructed to enable standardized mucus administration. (B) Lungs are positioned at an angle using the tilt table prior to mucus delivery. (C) Tilt table features key design components including sterile surface for contact with lungs, hook to secure the carina during tilting, and radiolucent flexible lung support. (D) Mucus is delivered to the airways under bronchoscopic guidance. (E) Bronchoscope is visualized on radiography and mucus is instilled at a continuous rate via peristaltic pump to ensure uniform coating of the airways. (F) Mucus coating the airways directly visualized following delivery via dissection.

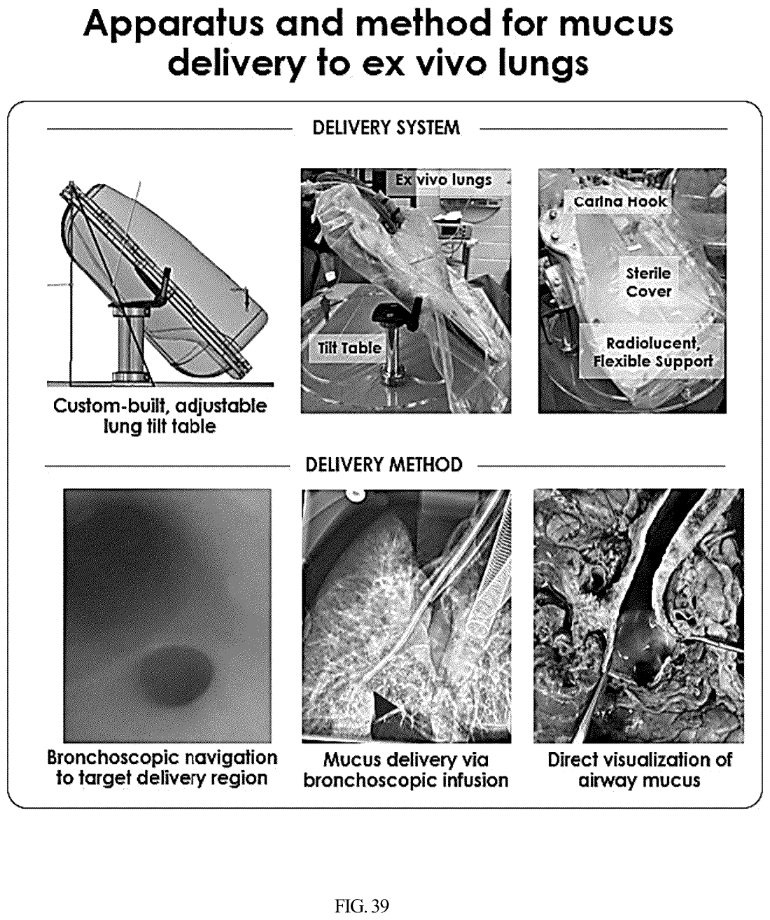

FIG. 40: Bronchoscopic visualization of mucus delivery and maintenance during cross-circulation. (A) Time course of mucus instillation after visualizing the airways. Bubble in airway shows mucus plug advancing through airway generations. (B) Mucus is maintained in airways on cross-circulation throughout 24 hours.

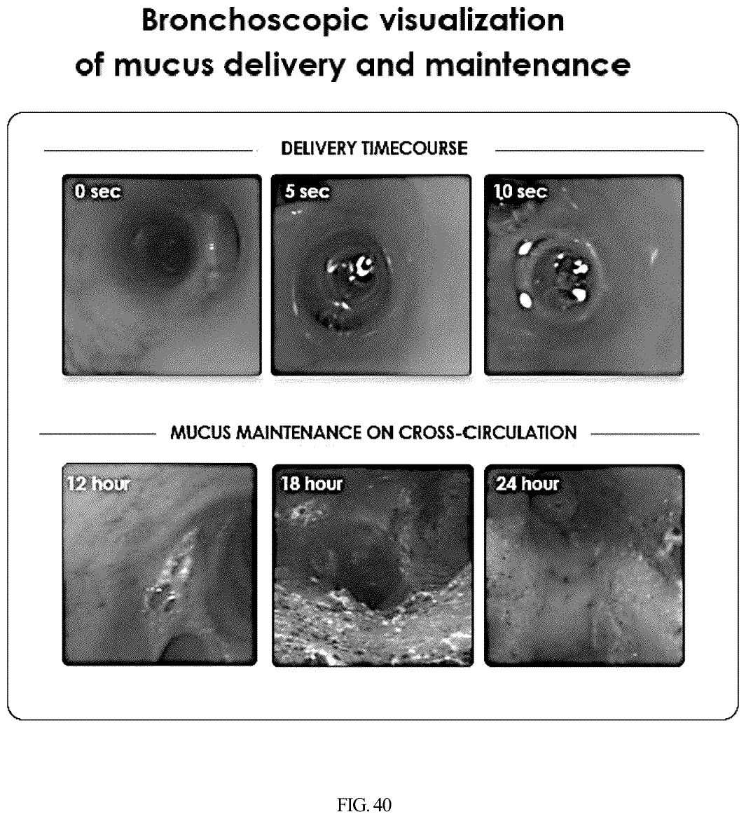

FIG. 41: Lung tissue viability following mucus delivery. (A) Metabolic activity of parenchymal tissue with and without mucus confirms tissue viability (B) CFSE uptake in airway tissue at baseline and 24 hours following mucus delivery.

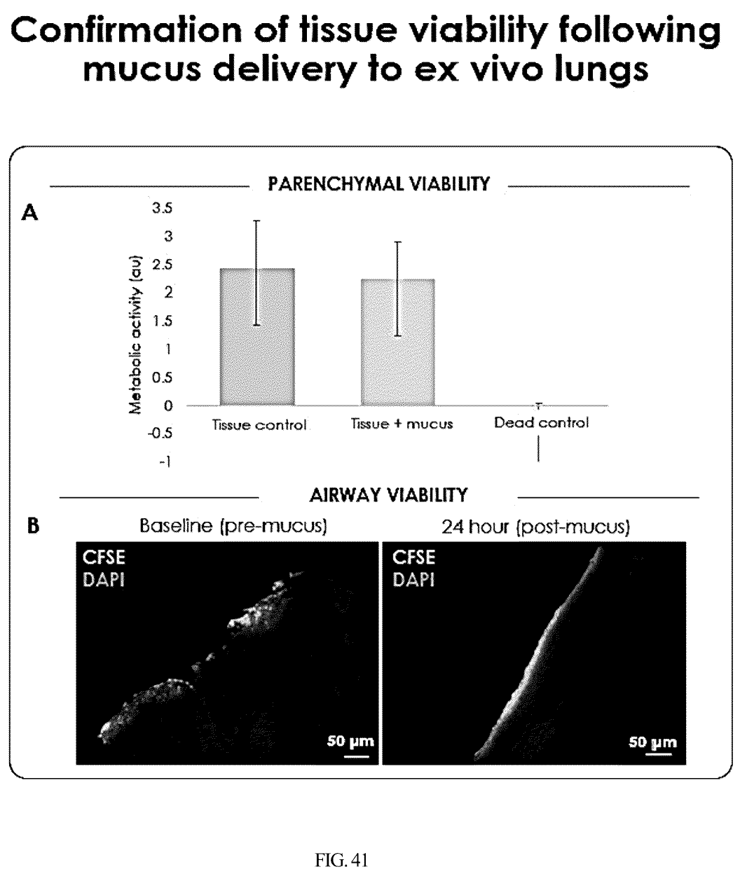

FIG. 42: Radiographic visualization of mucus delivered to ex vivo airways.

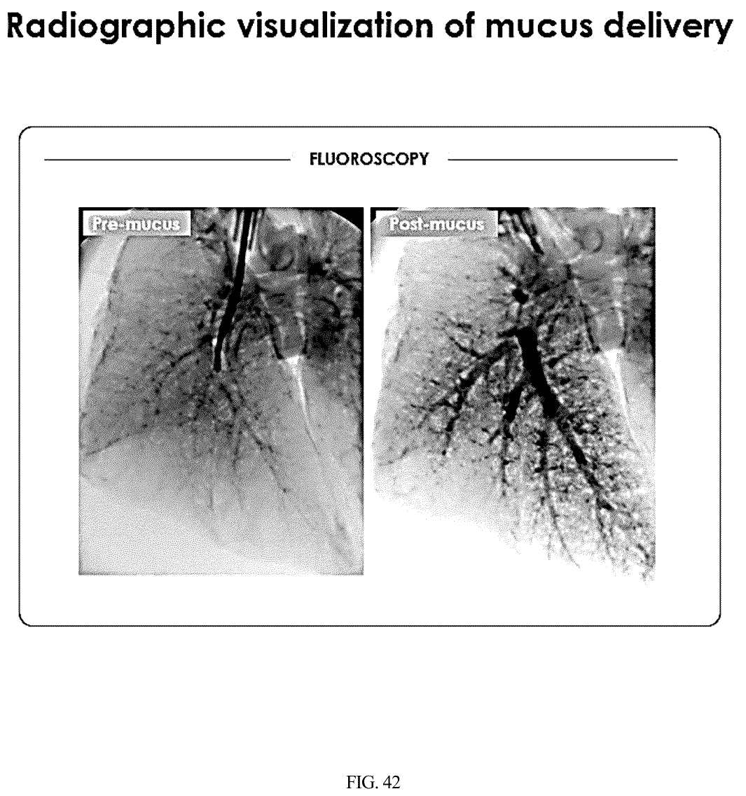

FIG. 43: Histologic visualization of mucus maintained in airways 24-hours following delivery on cross-circulation.

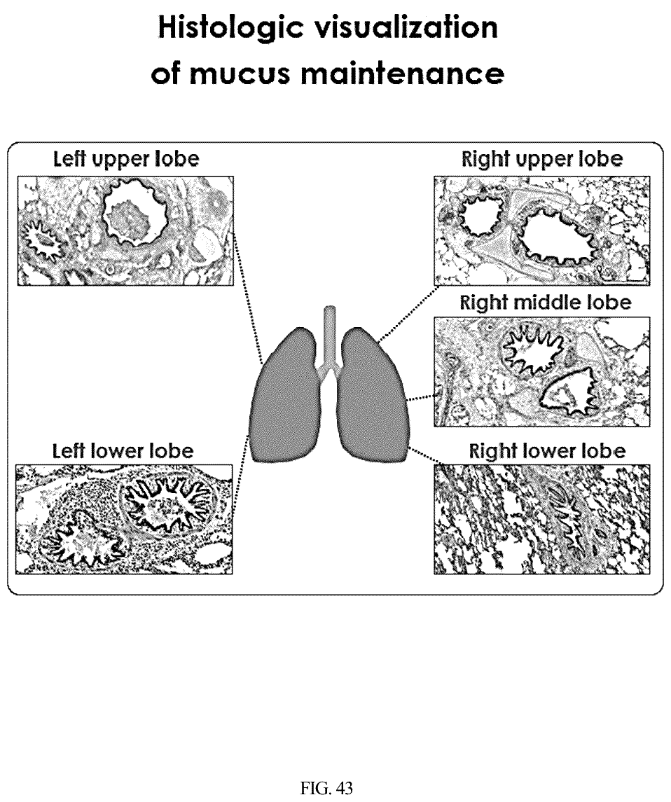

FIG. 44: Transpleural injection of nanoparticles for delivery to ex vivo lungs.

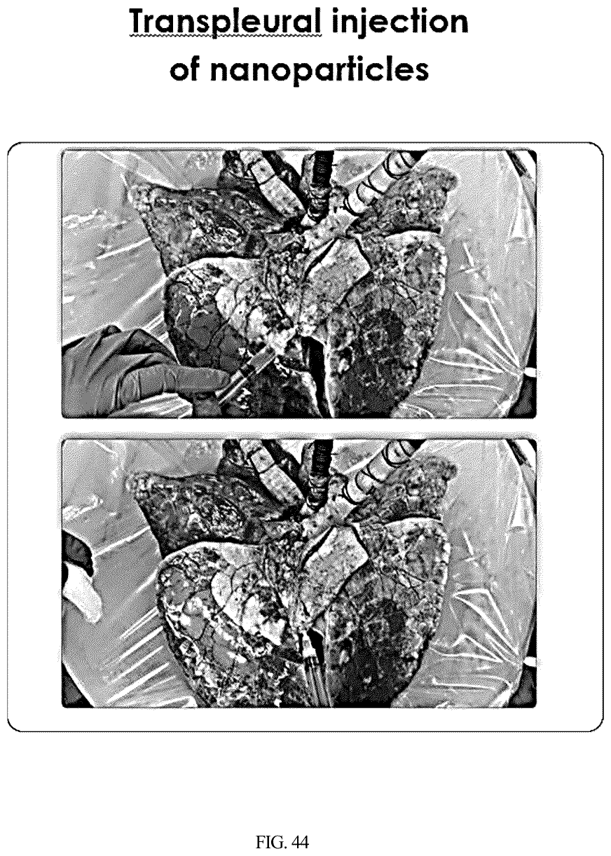

FIG. 45: Bronchoscopic delivery of nanoparticles using catheters in the ex vivo airway. (A) Catheter placement via bronchoscope (blue arrows). (B) Nanoparticle delivery (Red arrows). (C) Bronchoscopic visualization of airway catheter. (D) Nanoparticles visualizable using airway bronchoscopy.

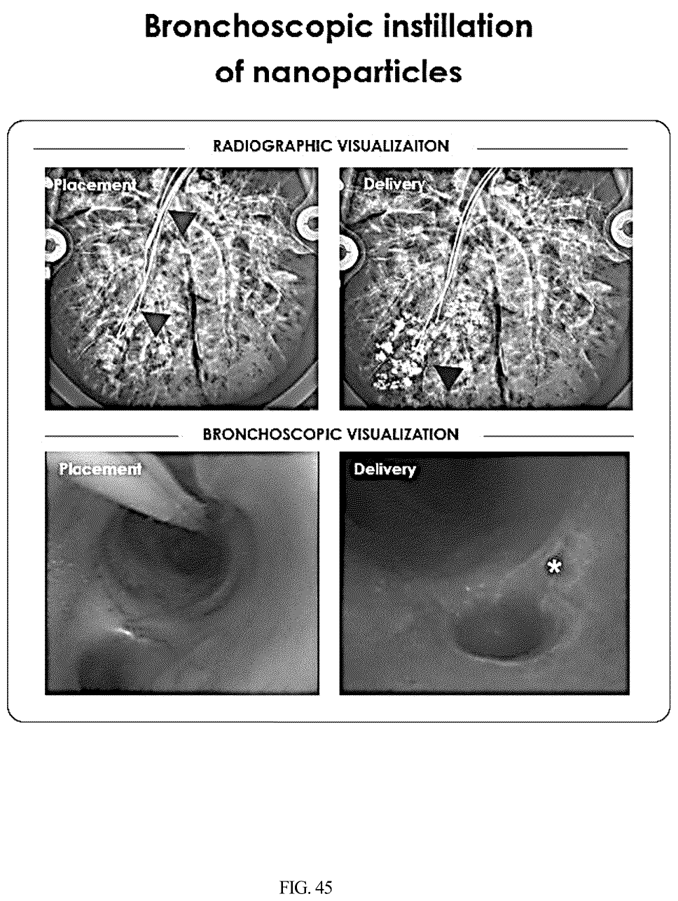

FIG. 46: Nebulization of nanoparticles to ex vivo lungs. Ex vivo nebulization allows administration of clinically relevant doses of drugs, molecules, and genes.

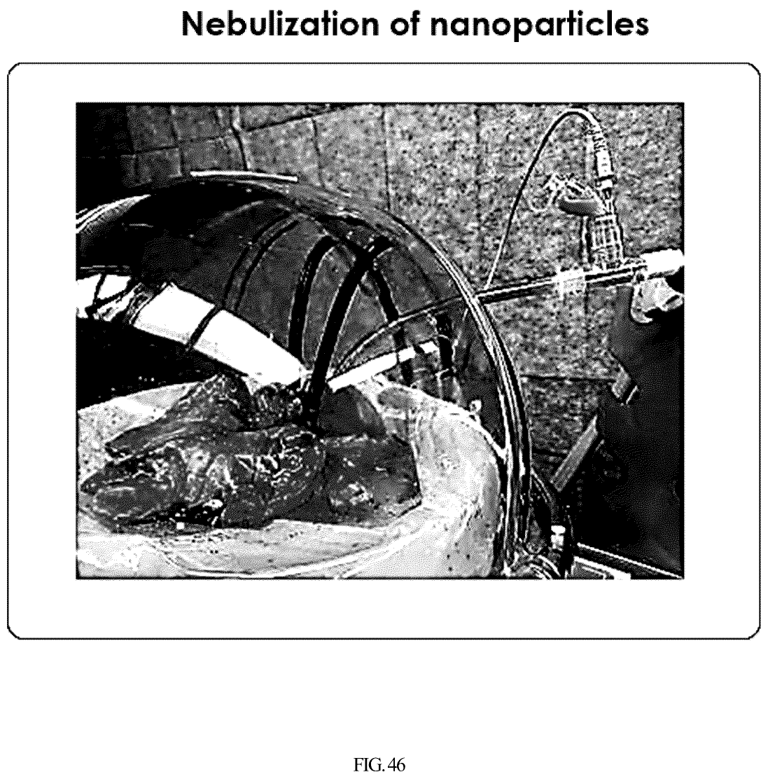

FIG. 47: Expression of protein following mRNA delivery in lipid nanoparticles. mCherry and eGFP protein expression were observed 12 hours following lipid nanoparticle delivery containing mCherry and eGFP mRNA in ex vivo lungs.

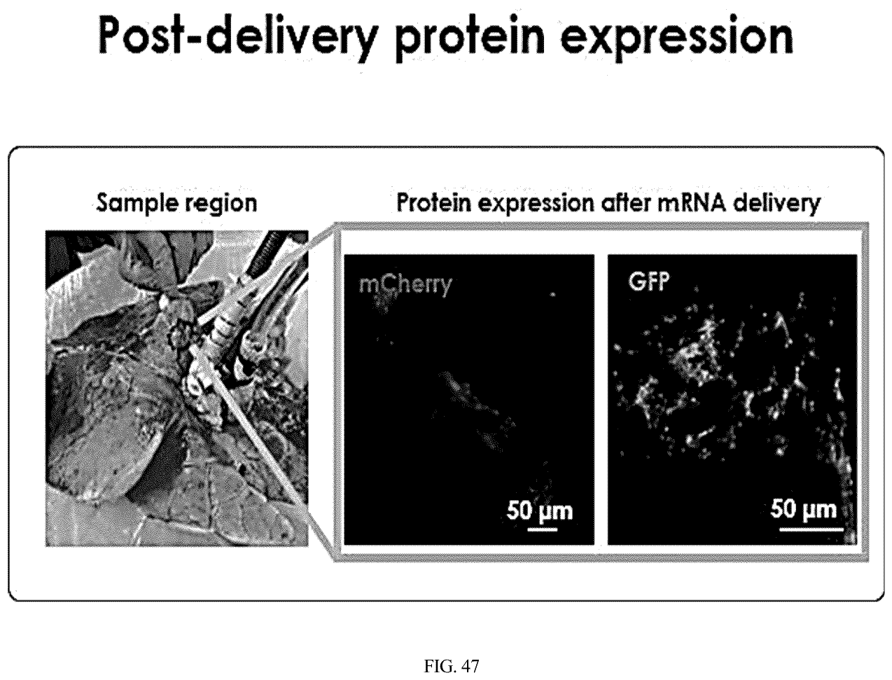

### DETAILED DESCRIPTION

A mucosal tissue bioreactor comprising:
a base comprising one or more wells that support a culture medium;
one or more platforms configured to support a portion of an airway mucosa in at least partial contact with the culture medium in the one or more wells;
a fluid circulation loop that circulates the culture medium through the one or more wells; and an air circulation loop that circulates air over a top surface of the portion of the airway mucosa.

An air-liquid interface bioreactor with integrated perfusion and ventilation that supports a slice of mammalian airway mucosa in culture comprising:
a base comprising one or more wells for holding a culture medium;
one or more platforms for supporting the mucosa in contact with the culture medium in the one or more wells;
a fluid circulation loop for circulating culture medium through the one or more wells; and
an air circulation loop for circulating air over a top surface of the mucosa.

In embodiments, the one or more wells are surrounded on a horizontal plane thereof by vertical walls so as to keep fluid in said wells within wells.

In embodiments, each of the one or more wells comprises an entry side and a removal side separated by a half wall that has a height smaller than a height of remaining walls of the well; wherein the entry side is in fluid communication with inlet tubing of the fluid circulation loop; and the removal side is in fluid communication with outlet tubing of the fluid circulation loop so that culture medium level is maintained at the height of the half-wall on the entry side.

In embodiments, the one or more platforms comprise a chair-like tissue holder comprising a polymeric frame with a permeable mesh seat on which the portion of the airway mucosa is positioned and one or more legs, the legs having a long axis substantially perpendicular to a plane of the permeable mesh seat.

In embodiments, the portion of the airway mucosa is attached to the permeable seat portion of the chair-like tissue holder with a collagen hydrogel or other adhesive.

In embodiments, the chair-like tissue holder comprises four legs, which plug into complementarily-shaped holes in a floor of the reactor so as to stabilize the holder's position.

In embodiments, the permeable mesh seat of the chair-like tissue holder is disposed near the height of the culture medium in the one or more wells so that the portion of the airway mucosa positioned thereon is maintained in an air-liquid interface wherein a top surface of the portion of the airway mucosa is exposed to air and a bottom of the portion of the airway mucosa is submerged in the culture medium.

In embodiments, an airflow is introduced to the top surface of the portion of the airway mucosa and a perfusate is introduced to the submerged bottom of the portion of the airway mucosa.

In embodiments, the bioreactor comprises:
a lid comprising a central opening above the location of the tissue in its chair-like tissue holder(s), wherein the opening is optionally:
left open to the air for easy loading and removal of the entire mucosa airway slice and/or chair-like tissue holder;
sealed with a coverslip and adhesive;
sealed with an insert that holds a recessed coverslip deeper in the chamber than the remainder of the lid for the purpose of bringing imaging equipment in closer proximity to the surface of the tissue;
sealed with an insert that comprises a top window comprising a transparent material and/or material compatible with visual microscopy.

In embodiments, the bioreactor body substantially comprises one or more PTFE plastics, or one or more polycarbonate plastics.

In embodiments, the lid is detachable, and is securable by one or more pins, wherein an airtight seal is formed when the lid is secured by one or more pins.

In embodiments, the lid is securable by two pins, positioned on opposite outside ends of outside surfaces of walls of the bioreactor.

In embodiments, the bioreactor comprises one or more windows in a side wall of the reactor positioned such that a side profile of the portion of the airway mucosa, tissue holder, culture medium below the portion of the airway mucosa and mucus/air above the portion of the airway mucosa are observable.

In embodiments, the bioreactor further comprises a peristaltic pump and a heating system employing a heating pad with a PID controller and digital readout.

In embodiments, the bioreactor body substantially comprises an epifluorescence-compatible and light sheet microscopy-compatible material.

In embodiments, the bioreactor the top window comprises epifluorescence-compatible and/or light sheet microscopy-compatible material.

In embodiments, the well(s) comprise an upper and lower compartment (e.g., in a non-limiting example, 6 mm high x 7 mm wide x 2 cm long inner compartment, with a central 5 mm diameter hole connecting the upper and lower chambers), into which the airflow and perfusate can be introduced, respectively. In embodiments, the two compartments can be separated by a porous polycarbonate membrane bridging the central connecting hole (e.g., pore size 0.3 μm to 0.5 μm, such as 0.4 μm; for example, Nunc™ Cell Culture Inserts, Thermo Scientific™), onto which the tissue slices will be placed with their luminal surface facing the airspace. In embodiments, the upper (airspace) side of the membrane can be coated with, e.g., 4% (w/v) alginate gel (e.g. thickness: 1 mm) before the tissue loading to secure the tissue slices within the channel and prevent leakage of perfusate into the airflow channel. This arrangement will allow tight control of physio-mimetic air-liquid interface at the airway lumen that is important to promote tissue viability. In embodiments, multi-way connectors (Masterflex Fitting, Cole-Parmer) can be attached to the inlets and outlets of both airflow and perfusion channels via polymer tubing (Tygon® tubing, Cole-Parmer). Airflow can be be controlled via a small animal ventilator (e.g., PhysioSuite, Kent Scientific), and perfusion flow can be provided via a peristaltic pump (e.g., L/S® digital pump system, Cole-Parmer), or both airflow and perfusion flow can be supplied by miniature peristaltic pumps (e.g. Bartels mp6 micropumps).

A plurality of bioreactors as described herein, each comprising a portion of an airway mucosa slice.

In embodiments, the bioreactor the plurality of bioreactors are maintained in parallel in a standard tissue incubator.

In embodiments, the bioreactor further comprises the portion of the airway mucosa supported on the platform.

In embodiments, the bioreactor further comprises bioartificial mucus on a top surface of the portion of the airway mucosa.

In embodiments, the portion of the airway mucosa comprises at least one submucosal layer.

In embodiments, the portion of the airway mucosa comprises ciliated columnar epithelium, connective tissue, and submucosal glands.

In embodiments, the portion of the airway mucosa slice comprises trachea. In embodiments, the airway mucosa slice comprises bronchi or bronchioles.

In embodiments, the bioreactor supports a portion of the airway mucosa having a size of 600 microns or less.

In embodiments, air is circulated over a top surface of the portion of the airway mucosa so as to match at least one of a breathing rate or tidal volume matched to flow parameters in an upper, mid, or lower airway from a host animal type from which the portion of the airway mucosa has been obtained.

A bioartificial mucus benchmarked to a patient sputum in macromolecular composition, rheology, dextran permeability, and microrheology via particle tracking. In embodiments the bioartificial mucus comprises DNA, phospholipids, and mucin to create a solution that mimics CF mucus.

In embodiments, in terms of macro-rheology, the bioartificial mucus matches CF patient mucus. In embodiments, the bioartificial mucus has a frequency-dependent elastic modulus (G′) of about 7.25-7.35 Pascal (average). In embodiments, the bioartificial mucus has a frequency-dependent viscous modulus (G″) of about=2.1-2.3 Pascal (average). In embodiments, the bioartificial mucus has a shear viscosity (eta) of about=50-52 Pascal*seconds (average). In embodiments, the bioartificial mucus has a frequency-dependent elastic modulus (G′) of about 7.3 Pascal (average). In embodiments, the bioartificial mucus has a frequency-dependent viscous modulus (G″) of about=2.2 Pascal (average). In embodiments, the bioartificial mucus has a shear viscosity (eta) of about=51 Pascal*seconds (average).

In embodiments, in terms of micro-rheology, the bioartificial mucus has a reduction in mean squared displacement by at least three orders of magnitude (10\hat{ }3) for standard nanoparticle probes diffusing in the bioartificial mucus preparation vs. water. In embodiments the diffusion coefficients for standard nanoparticle probes in the bioartificial mucus vs. patient mucus are of the same order of magnitude.

A whole lung model of cystic fibrosis comprising
an ex vivo lung supported with a cross-circulation apparatus,
bioartificial mucus present in the ex vivo lung airways which mimics mucus of cystic integrated real-time imaging and/or analytic tools for assessment of lung function and therapeutic uptake and efficacy.

In embodiments, the cross-circulation apparatus comprises:
an extracorporeal organ chamber for containing and supporting the ex vivo lung, wherein the chamber comprises a time- and temperature-controlled humidifier and misting spray onto the ex vivo lung and into the chamber to maintain a humid environment within the chamber; and an extracorporeal cross-circulation circuit for connecting ex vivo lung with a host organism for cross-circulation of blood or plasma, which circuit comprises:
fluid conduits providing flow of the blood or plasma between a vascular system of the host organism to vasculature of the ex vivo lung;
an arterial pressure sensor for sensing extracorporeal in-flow line pressure;
a venous pressure sensor for sensing extracorporeal out-flow line pressure; and
a circulating pump which regulates blood or plasma flow within a predetermined percentage range of cardiac output of the host organism based on the trans-organ pressure difference between arterial and venous pressure measured by the arterial pressure sensor and the venous pressure sensor.

In embodiments, the system comprises an ex vivo lung obtained from a human donor lung or from swine lungs. In embodiments, the system comprises an ex vivo lung obtained from a human donor lung with cystic fibrosis.

In embodiments, the bioartificial mucus comprises mucin(s), DNA, erythrocyte ghosts, lipids, albumin, water. In embodiments, the bioartificial mucus is introduced into the airways of the ex vivo lung via image-guided bronchoscopic infusion. In embodiments, the bioartificial mucus is present as a circumferential thin film throughout one or more airways.

In embodiments, the integrated real-time imaging and/or analytic tools can monitor properties of exogenous mucus, lung function, cellular expression, therapeutic distribution, therapeutic uptake, and/or therapeutic efficacy.

In embodiments, the integrated real-time imaging and/or analytic tools comprises one or more of an airway pressure measurement and airway volume measurement, radiographic measurement, thermographic measurement, bronchoscopic imaging, transpleural fluorescent scope, and/or a bioluminescent imaging scope.

In embodiments, the bioreactor further comprises one or more electrode probes to measure airway tissue impedance.

In embodiments, blood or plasma flow is kept within the range of 5% to 10% of cardiac output of the host organism.

A method of ex vivo screening of a candidate therapeutic for Cystic Fibrosis comprising administering the candidate therapeutic to the lung in the system described herein and determining one or more of candidate therapeutic uptake, distribution, and efficacy in real-time.

A method of ex vivo screening of a candidate therapeutic for Cystic Fibrosis comprising administering the candidate therapeutic to the lung in the system described herein and determining a status of the lung before and after administration of the candidate therapeutic for Cystic Fibrosis so as to determine if the candidate therapeutic improves one or more symptoms of Cystic Fibrosis.

The term “about” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Where particular values are described in the application and claims, unless otherwise stated, the term “about” meaning within an acceptable error range for the particular value should be assumed.

### Example 1

We developed a physiologic airway mucosa and parenchyma bioreactor for disease modeling and therapeutic screening. We hypothesized that by developing a bioreactor that could preserve lung tissue outside the body for a period of time, we could test new drug delivery strategies to find a better way to case or prevent the symptoms of CF.

We used Autodesk Fusion 360, SolidWorks, and UltiMaker Cura to rapidly prototype a bioreactor that supports a small slice of lung tissue under ventilation. We built a proof-of-concept perfusion system using a peristaltic pump, and a heating system using a heating pad with a PID controller and digital readout. We combined DNA, phospholipids, and mucin to create a solution that mimics CF mucus.

We describe herein a mucosal tissue bioreactor for ex vivo culture of airway tissues, modeling of disease, and therapeutic screening. One envisioned application of this device is to model cystic fibrosis (CF) for the purpose of drug and gene therapy testing (i.e., CF-mimetic airway model), though the platform could be extended to other airway and lung disease applications. The CF-mimetic airway model is comprised of: (i) integrated image-enabled airway tissue bioreactor and (ii) bioartificial CF-mimetic mucus that replicates transport phenomena found in the CF airway.

We developed an opto-electrically integrated bioreactor platform to create a CF-mimetic in vitro airway model with adjustable biophysical characteristics to resemble the environment within the human CF airways, such as mucus thickness, viscosity, and hydration. This CF-mimetic airway model may be used to investigate how the mucus formulation and the dose and properties of therapeutics (e.g., particles loaded with genetic cargo) influence internalization and endosomal escape of the cargo. The CF-mimetic airway model may be used to investigate whether transient disruption of the mucosal-epithelial barrier may increase the amount and speed of therapeutic uptake into lung cells. The CF-mimetic airway model may incorporate mucosal tissue slices obtained from different anatomic regions (e.g., trachea, bronchi, bronchioles) of mammalian lungs, e.g., porcine, human lungs. Isolated airway tissue supported in the bioreactor may then be supplemented with the bioartificial CF-mimetic mucus to replicate the properties of the airway surface found in vivo. To achieve real-time and non-invasive tracking and quantification of delivered therapeutics in screening applications, we designed the bioreactor to be compatible with light sheet microscopy and integrated with other analytic modalities including bioimpedance measurement, tissue stiffness quantification, among other capabilities.

Construction of mucosal airway tissue bioreactor platform: We created a flow-controlled airway tissue bioreactor, coupled with our customized optical and electrical measurement systems, such as a light-sheet microscope and bioimpedance analyzer. The bioreactor chamber channel network may comprise a single internal compartment into which the airflow and perfusate are introduced to mimic ventilation and perfusion, respectively.

Perfusion and ventilation setup. Unidirectional media flow with control of media height is produced as follows: Media flow is introduced through tubing that opens just above the floor of the reactor on the “entry” side, and media is allowed to fill the entry side of the reactor until it spills over a small half-wall into the “removal” side. On the removal side, a second downward-facing tube that opens just above the level of the reactor floor serves to remove excess media which has flowed over the internal half-wall. A peristaltic pump drives media recirculation between these two halves of the chamber. In this way, media level is maintained at the height of the half-wall on the entry side. Airflow is controlled by a separate peristaltic pump, and is circulated by tubing entering and exiting near the top of the single open chamber. Airflow may be in the same direction or a different direction from media flow. Multi-way connectors may be attached to the inlets and outlets of both airflow and perfusion channels via polymer tubing. The airflow and perfusion may be independently controlled with a ventilator and peristaltic pump with integrated real-time feedback.

Tissue holder and maintenance of tissue in air-liquid interface. On the media entry side, just before the half-wall, is positioned a chair-like tissue holder, consisting of a polymeric frame with a permeable mesh “seat”, on which tissue slices may be placed. The tissue slices may be attached to the seat with collagen hydrogels or other adhesive measures. The legs of the chair-like tissue holder plug into complementarily designed holes in the floor of the reactor to stabilize the holder's position. This maintains the “seat” of the chair near the height of the media in such a way that the tissue is maintained in an air-liquid interface (top of tissue in air, bottom of tissue/tissue holder submerged in media).

Tissue access and imaging windows. The lid of the reactor has a central opening just above the location of the tissue in its chair-like holder. This opening may be 1) left open to the air, for easy loading and removal of the entire tissue and/or chair-like tissue holder, 2) sealed with a coverslip and adhesive, 3) sealed with a custom insert that holds a recessed coverslip deeper in the chamber for the purpose of bringing imaging equipment in closer proximity to the surface of the tissue, or other custom inserts as designed to be compatible with our imaging platforms. There is a second window, with the potential of more than one window, in the side wall of the reactor that is positioned such that the side profile of the tissue, tissue holder, media below, and mucus/air above the tissue are visible with our imaging devices.

Isolation and sterility. The reactor may remain open to the air through the central roof opening and maintained within secondary containment for steady state culture. The reactor may also be scaled so that it is airtight and water-tight, with a 0.2 micron or smaller filter connecting the aerated portion to outside air, either at the point of the roof of the bioreactor or at any point along the ventilation circuit. To contain any potential infectious/genetic cargo-carrying agents, the source of external air may itself be a large sealed container (i.e., a large volume of air to be recirculated to the tissue without being open to the atmosphere). The entire bioreactor may be sterilized and is compatible with a standard cell culture incubator.

Creation of CF-mimetic airway tissues: Using this platform, we created CF-mimetic airway tissues from mucosa obtained from freshly explanted lungs of euthanized Yorkshire swine and human donor lungs (from lung transplant programs) and our custom CF-mimetic bioartificial mucus formulation.

Tissue preparation. While porcine tissues may be used for most studies, human donor lungs may be used to replicate a subset of studies, and the results may be compared. We may also obtain mucosa from explanted lungs of CF piglets when available to compare with findings in non-CF swine and human lungs. We aim to validate porcine tissue as a model of human tissue, and elucidate any differences between the tissue types. Airway tissues may be harvested from different regions of the lung (e.g., upper, mid, or lower airways) and sectioned. Cylindrical tissue slices (diameter: 1-5 mm, thickness: 0.1-2 mm), with or without washing in PBS, may be obtained and transferred into the bioreactor. As described above, collagen gel may be coated onto the porous membrane of the chair-like tissue holder's “seat” prior to the placement of the tissue slice. The perfusion channel may be filled ahead of time with cell culture medium and the airflow channel may be ventilated (breathing rate: 10-20 bpm; tidal volume: matched to flow parameters in upper, mid, and lower airways). When ready for use, the tissue may be installed through the superior opening in the roof of the reactor, and the roof finished with one of the three methods mentioned above (left open, sealed with coverslip, sealed with custom insert).

Bioartificial mucus preparation and instillation. Bioartificial mucus may be prepared from a combination of biological ingredients inspired by our characterization of cystic fibrosis patient mucus as well as literature reported components and exogenous crosslinking agents and other agents as necessary to achieve the biophysical and chemical properties matching patient sputum. In some cases, actual patient sputum may be used. We place a small bolus of mucus (volume dependent on mucus viscosity and capillary number) onto the airway slice to create a mucus layer before or after loading tissue; mucus layer may be replaced through instillation through dedicated tubing entering through a dedicated roof insert via our previously established plug flow film generation technique. The device may be ventilated with ambient air for 5 min to improve mucus adhesion onto the airway surface. To create thicker mucus film (>100 μm), the bolus instillation procedure may be repeated. The correlation between mucus delivery and thickness may be evaluated via microscopy of the airway cross-section following the mucus deposition. Viscoelastic physical properties of the deposited mucus may be measured by our custom-built vacuum-based rheometer directly on the airway surface.

The bioartificial mucus can be formulated, broadly, from a combination of: 1) a deoxyribonucleic acid (DNA) source, 2) a mucin protein source, 3) other protein sources, especially sources of serum proteins; 4) a lipid source, 5) macromolecules to imitate bacterial biofilm composition, 6) exogenous crosslinking agents, 7) water, 8) biologically relevant salts, 9) iron chelators. Individual components can be omitted if desired. In particular:

Deoxyribonucleic acid (DNA): preferably in a strand form that is 2000 base pairs or longer. Source may include DNA extracted from salmon testes (commercially available). In embodiments, the concentration range is approximately 0.16-5.60 mg/mL but can be expanded outside this range to imitate atypical/outlier patient samples.

Mucin protein: may be obtained commercially, as from porcine gastric mucosa or from bovine submaxillary gland. Ideally, the mucin protein is obtained from porcine gastric mucosa or from porcine lung tissue, extracted via in-house procedures. In embodiments, the concentration range is approximately 7.9-35 mg/mL but can be expanded outside this range to imitate patient samples.

Other protein sources: Can include fetal bovine serum or purified bovine serum albumin, as well as other proteins as identified to be important based on our studies of cystic fibrosis patient sputum. In embodiments, the concentration range is approximately 0-65.1 mg/mL.

Lipid content: In one formulation, lipids will be sourced from egg yolk phospholipids, either sourced commercially or extracted in-house. In another formulation, lipid content will be primarily provided by cells and/or cellular components, as provided by red blood cell ghosts (that is, red blood cells that have been chemically treated so as to remove their internal hemoglobin and other proteins, leaving behind the empty and intact cell membrane), or from purified cellular debris from homogenized lung tissue. A combination of the sources may also be used. In embodiments, the concentration range is approximately 25.3-41.7 mg/mL, but may range outside of this to imitate patient samples.

Macromolecules to imitate bacterial biofilm composition: These may include alginate and rhamnolipids, and would be supplied at concentrations of less than 2% by weight each.

Exogenous crosslinking agents: These can be included to achieve the desired mechanical properties. These may include chemistries such as EDC and N-hydroxysulfosuccinimide (NHS), periodate/norbornene/tetrazine, glutaraldehyde, genipin, or transglutaminase, along with other small molecules that may be required to provide the required chemical groups to establish these crosslinking mechanisms. Their concentrations will range widely depending on the chemistry, but in embodiments, are 0-1% by weight.

Water: distilled, deionized, or ultrapure water will be included at the volume required to prepare a desired volume of mucus.

Biologically relevant salts: may include sodium chloride (approximately 0.9% wt or higher), potassium chloride, calcium salts, magnesium salts, and phosphates.

Iron chelators: such as diethylene triamine pentaacetic acid (DTPA), at a concentration of approximately 0 or 5.9 mg/L, but may vary.

We designed a platform to support native mucosal tissues with high throughput capabilities for medium-term culture and unique capability of supporting native human/porcine tissues. Preliminary work demonstrated tissue viability and functionality at 7 days and usability with a wide array of noninvasive measurement/detection systems.

We designed a bioartificial mucus that mimics the CF airway extracellular environment and forms a drug delivery barrier and pathogen-protecting agent, which may be useful for developing therapies for HEMT-nonresponsive CF patients.

Bioartificial mucus with low macroscopic viscosity still has significant barrier properties, causing aggregation and up to 68% decrease in mean squared displacement at one second (MSD(1s)) of 100 nm polar probes, and up to 39% decrease in MSD(1s) of 100 nm PEGylated probes. Recovered mucosal layers show minimal epithelial or basement membrane disruption on histology and remain viable in mucosal tissue bioreactors.

Our prototype demonstrates proof-of-concept for a self-sustaining lung tissue slice bioreactor for studying CF. Although our artificial CF mucus was not as viscous as CF mucus, this formulation may be refined in the future.

This work has demonstrated proof-of-concept for an air-liquid tissue bioreactor with artificial CF mucus for studying new treatments for CF.

Although the bioreactor and bioartificial mucus are described herein for culturing and studying lung mucosa, the embodiments described herein may be adapted for disease modeling and drug screening in mucosal tissues other than lung, including gastrointestinal, nasal, oral and vaginal applications.

### Example 2

This subject matter is directed to a whole lung model of cystic fibrosis made up of (i) an ex vivo lung supported with a cross-circulation bioreactor, (ii) bioartificial mucus delivered into the ex vivo lung airways to mimic transport conditions found in cystic fibrosis, (iii) integrated real-time imaging and analytic tools for assessment of lung function and therapeutic uptake and efficacy.

The cross-circulation bioreactor supports ex vivo lungs. The ex vivo lung can be obtained from either human donor lungs (CF or normal) or from swine lungs. The cross-circulation bioreactor provides normothermic perfusion with whole blood via a swine ‘host’ connected to a modified extracorporeal membrane oxygenation (ECMO) circuit and humidified ventilation using a portable ventilator. The lungs may be modified to mimic CF by inducing airway injury through chemical, mechanical, or biological means; by administering drugs that cause production of mucus; or by introducing (ii) bioartificial CF-mimetic mucus.

Bioartificial CF-mimetic mucus is comprised of mucins, DNA, erythrocyte ghosts, lipids, albumin, ions at various concentrations, and other chemical and biological components. Additional components, such as radiopaque dye, can be added to the mucus for visualization. The bioartificial CF-mimetic mucus is specifically designed to match properties of mucus and sputum obtained from CF patients experiencing pulmonary exacerbations. The bioartificial CF-mimetic mucus is introduced into the airways of the ex vivo lungs via image-guided bronchoscopic infusion at a specified rate of infusion to generate a circumferential thin film throughout the airways. Confirmation of delivery can be performed via x-ray or bronchoscopy for direct visualization of mucus. Functional changes within the lung can be further analyzed using (iii) integrated real-time analytics.

The whole lung bioreactor is also integrated with real-time analytic tools to monitor properties of exogenous mucus, lung function, cellular expression, and therapeutic distribution, uptake, and efficacy. These tools include airway pressure and volume measurements; radiographic, thermographic, and bronchoscopic imaging; and transpleural fluorescent and bioluminescent imaging scopes.

Altogether, the whole lung model of CF addresses several major challenges in the development of therapeutics to treat cystic fibrosis. Existing models do not recapitulate the airway mechanics and delivery conditions found in the CF lung. Because of this, many pre-clinical models generate a large number of false positives which increases costs and leads to many therapeutics failing during clinical trials. To date, over 35 gene therapy trials for CF have failed. An ex vivo whole lung model of CF allows for ex vivo screening of therapeutics for CF in a clinically relevant environment that mimics the delivery conditions actually present in human CF lungs. The CF model is supported with a bioreactor that has integrated analytics for monitoring therapeutic uptake, distribution, and efficacy in real-time.

Disclosed is a high-fidelity, robust, and widely accessible CF model system that enables: i) predictive screening of gene or mRNA therapies, ii) optimization of therapeutic administration and delivery strategy including adjunct interventions to promote therapeutic uptake, and iii) understanding of surface properties that allow nanoparticles to penetrate the mucus barrier. The multi-scale bioengineered lungs, including in vitro airway tissue models and ex vivo whole porcine and human lungs, combined with tunable, bioartificial CF mucus can accurately model CF lungs, allowing high-throughput screening of candidate drugs, including nanotherapeutics.

A CF-mimetic airway tissue model for high-throughput in vitro investigation of nanoparticle transport and therapeutic effects: CF airway mucus is highly viscous compared with normal mucus, creating a primary physical barrier against gene therapies administered via the airway. Therefore, the unique biochemical and physical properties of CF mucus should be incorporated into the pre-clinical models of CF. We disclose a tunable bioartificial CF mucus and benchmark its properties against sputum samples obtained from CF patients being treated at the Gunnar Esiason Adult Cystic Fibrosis Center (Columbia University Medical Center). Bioartificial mucus is utilized for assessments of nanoparticle mobility to define optimal surface properties that promote mucus penetration. Further, we disclose incorporation of the bioartificial mucus into a 3D tissue model supported by our custom-built opto-electrically integrated bioreactor to investigate cellular uptake, endosomal escape, and therapeutic efficacy of locally administered nanoparticles.

A porcine CF-mimetic ex vivo porcine lung platform for evaluation of nanotherapeutic delivery, cellular uptake, and expression. In addition to mucus penetration, targeting delivery of inhaled or systemically-administered nanotherapeutics to appropriate regions of lung is a major challenge towards successful CF gene therapy. We apply the bioartificial mucus to whole porcine lungs to establish a human-size model of CF. Bioartificial mucus can be introduced bronchoscopically into porcine lungs, using liquid plug-flow models we previously developed. Nanotherapeutic administration and delivery (e.g., nebulized, aerosolized, spray-dried) will be evaluated in short-term (<12 hours) studies by measuring the distribution of nanoparticles into the appropriate airway regions in lungs on ex vivo lung perfusion (EVLP). Longer term studies (36-48 hours) using whole porcine lungs supported on cross-circulation will assess cellular uptake and expression of nanotherapeutic cargo by fluorescent and bioluminescent imaging and gene expression.

Evaluate nanotherapeutic uptake and expression in human lungs ex vivo. We l evaluate delivery methodologies and nanotherapeutic formulations using whole human lungs supported ex vivo. Because CF lungs are not routinely available for procurement, bioartificial mucus (mimicking human CF mucus) can be introduced into human donor lungs declined for transplantation and consented for use in research. We disclose utilizing optimized whole lung delivery methods and nanotherapeutics with the highest degree of uptake, as determined above. Importantly, results from human lung studies will serve as a benchmark for comparison with porcine lung studies to inform the fidelity of the porcine model. Delivery, cellular uptake, endosomal escape, and gene expression will be evaluated using new and existing methodologies developed by our group including bronchoscopic fiberoptic imaging, whole lung bioluminescent imaging, and transpleural fluorescent imaging.

Developing a CF-mimetic airway tissue model for high-throughput in vitro investigation of nanoparticle transport and therapeutic effects: Lack of animal models that faithfully mimic human CF lung has limited the development of nanoparticle-based gene delivery for treating CF. Small animal models fail to mirror the full pathophysiology of human CF. Porcine CF models can recapitulate human CF more closely, however, CF swine require intensive care as they have very low life expectancy (˜2 weeks). In vitro cultured airway tissues with physiologically relevant manifestation of CF would thus be highly valuable for optimizing and expediting gene therapy of CF.

Disclosed is a bioreactor platform that allows culture of CF-mimetic airway tissues for rapid evaluation of nanoparticle transport and intracellular gene delivery. This model integrates three innovative components that our team has been developing: i) bioartificial CF mucus with tunable biophysical properties; ii) computerized closed-loop bioreactor for cultivation and monitoring of porcine and human airway tissues; and iii) opto-electrically assisted real-time tracking of nanoparticles. Using this CF airway platform, we can correlate biophysical properties of mucus to the transport dynamics of various types of nanoparticles. We disclose systematically and longitudinally investigating dose-dependent rates of cellular uptake and endosomal escape of nanoparticles while determining the impact of transient disruption of the epithelial barrier on particle delivery into the airway basal stem cells (BSCs) using human and porcine tissues.

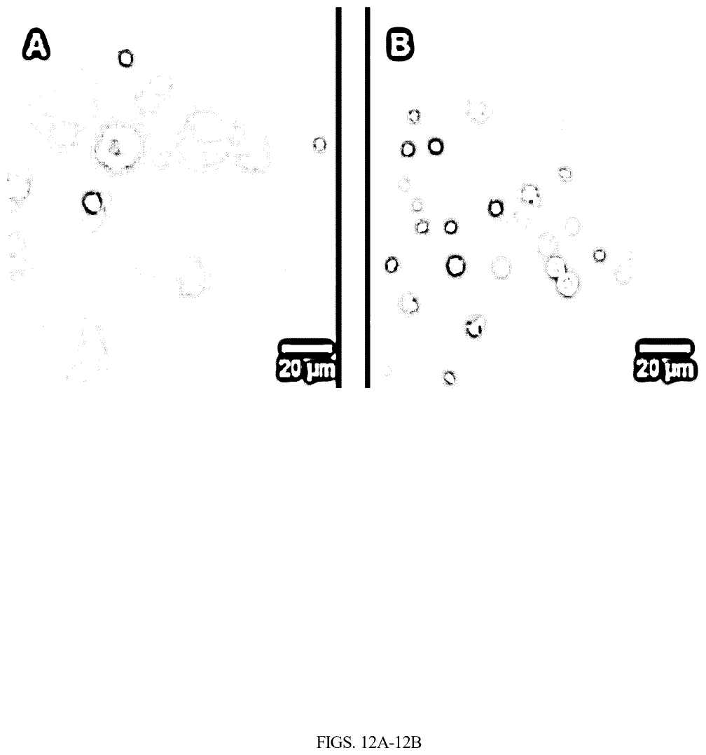

Results: Bioartificial mucus formulations and standardized nanoparticle tracking: Based on reported compositions of CF patient mucus (1), an existing artificial mucus formulation (2) was modified to contain DNA, lipids, carbohydrates, and proteins within ranges measured in CF mucus. These four variables can be arranged at three different levels (mean±s) to generate a panel of 81 mucus formulations representing the middle 68% of the CF mucus compositional range. We generated 9 of these 81 formulations and used them in preliminary nanoparticle tracking studies, as described below. Further modifications include removal of casamino acids (important to Pseudomonal growth but not to the biophysical features) and additions of fetal bovine serum (source of non-mucin protein), egg yolk phospholipids (for more accurate composition), and 1% penicillin-streptomycin (to comply with biosafety standards). In addition, preliminary particle tracking studies were conducted with 100-nm carboxylate-modified (negatively charged) and PEG-coated (polar uncharged) polystyrene nanoparticles, to measure particle diffusion for 9 formulations. Even in non-viscous formulations, the bioartificial mucus presented a biological barrier, especially to the travel of negatively charged particles, which moved slowly, sedimented, and aggregated. PEGylated particles, designed to resist aggregation and entrapment in mucus, better retained their normal diffusive behavior and were without apparent aggregation (FIG. 12, Table 1). Both the negatively charged and polar uncharged particles in bioartificial mucus followed their respective behaviors expected for patient mucus. These studies establish the feasibility of developing bioartificial mucus formulations matching CF patient mucus that can be used for nanoparticle mobility screening.

TABLE 1
Mean square displacements at a timescale of
1 second (MSDτ_{1 s}) for 100 nm nanoparticles.

| | | u − σ, | μ, | μ + σ, |
|---|---|---|---|---|
| | | DNA and | DNA and | DNA and |
| | H₂O | lipid | lipid | lipid |
| COOH polystyrene | 34.17 | 21.76 | 24.76 | 10.95 |
| (negatively charged) | | | | |
| PEGylated polystyrene | 31.97 | 30.39 | 19.44 | 23.25 |
| (polar uncharged) | | | | |

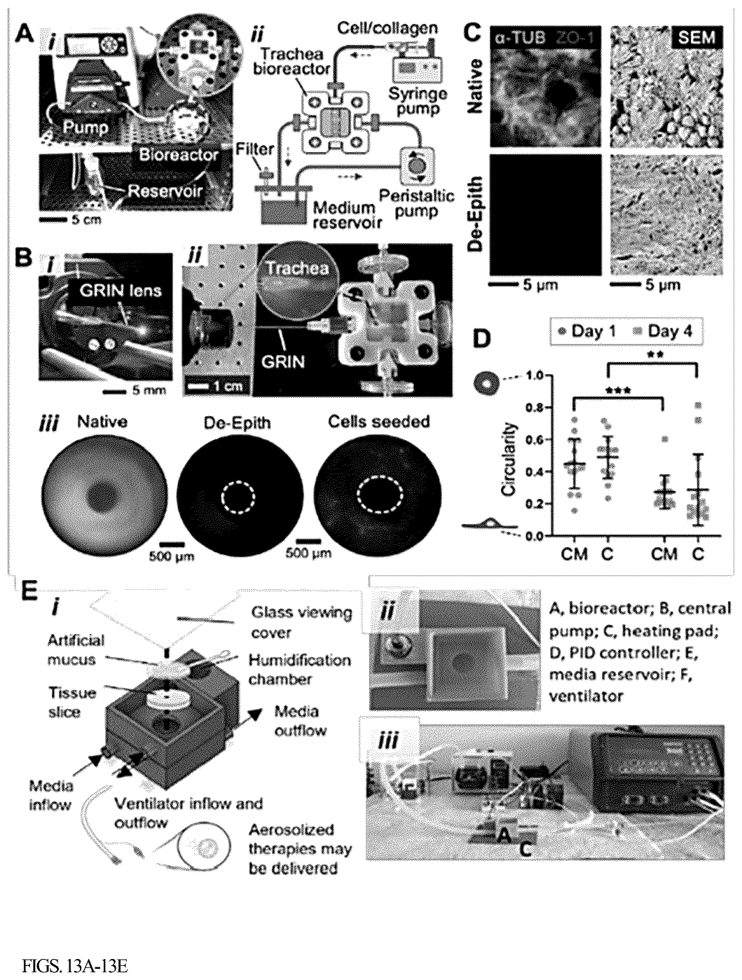

Imaging-enabled rat trachea bioreactor: We developed a flow-controlled bioreactor system that can allow imaging-guided manipulation and long-term cultivation of rat tracheal tissues (3-5) (FIG. 13). The bioreactor was constructed by integrating a tissue culture chamber (volume: 9.4 mL), a peristaltic pump (L/S® digital pump, Cole-Parmer), a syringe pump (AL-4000, WPI), and a culture medium reservoir, all of which were placed in an incubator (Isotemp™ CO2 Incubator, Thermo Fisher Scientific) (FIG. 13A). Using a gradient-index (GRIN) imaging lens (SELFOC®, NSG Group; diameter: 500 μm), in situ imaging of the airway interior was achieved at the cellular level (FIG. 13B, i-ii). Using this system, we demonstrated cell replacement in the rat trachea ex vivo. Lung epithelium was fluorescently labeled with carboxyfluorescein succinimidyl ester (CFSE; green) to visually confirm de-epithelialization following treatment with detergent (sodium dodecyl sulfate, SDS). Implanted cells were labeled with red quantum dots (emission: 655 nm) to monitor their distribution across the airway lumen (FIG. 13B, iii). Whole-tissue immunostaining of the untreated (native) rat trachea showed abundant cilia and tight junctions indicated, respectively, by acetylated alpha tubulin (a-Tub, green) and Zonula Occludens-1 (ZO-1, red), while de-epithelialized tissue lacked these components (FIG. 13C). The cell circularity (the ratio of the area to the perimeter of the cells) was measured as an indicator of cell engraftment (FIG. 13D). The seeded cells displayed a reduced circularity by day 4, suggesting that the bioreactor provides a supportive environment for cell engraftment, survival, and proliferation. Further, towards therapeutic testing of human-sized airway tissues, we generated a rapid prototype of a mucosal tissue bioreactor (FIG. 13E, i-ii), which was designed to be supported by similar ventilation and perfusion circuits as in the rat trachea bioreactor (FIG. 13E, iii). This bioreactor is capable of serving as a single module in a cassette of N units used for parallel testing of candidate nanoparticles.

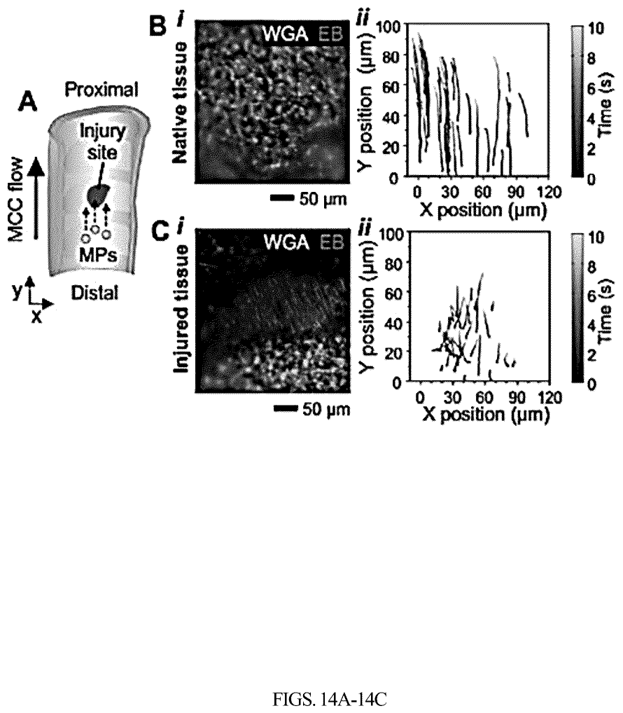

Quantification of mucociliary flow via particle tracing: We investigated the effect of epithelium injury on disruption of the mucociliary flow in rat airway tissues (FIG. 14). Real-time visualization of the epithelial integrity and cilia function was achieved by staining the cilia of freshly isolated airway tissues with fluorescein-labeled wheat germ agglutinin (FITC-WGA). While native airway tissue showed abundant cilia that continuously beat (FIG. 14B), fibrous connective tissue that was stained with Evans blue (EB) was observed at the injured tissue region (FIG. 14C). The speed and direction of the mucociliary flow at both acutely injured and native tissue regions were monitored by tracking 1-μm microparticles using a fluorescent microscope.

The particle trajectories revealed that the microparticles at the injured epithelial surface moved shorter distances than the microparticles at the ciliated tissue surface over a given amount of time (e.g., 10 s). The average speed of the microparticles measured at the injured region of damaged airway tissue (2.93±1.15 μm/s) was approximately 60% lower than those from the native tissue (7.1±1.1 μm/s) (p<0.001).

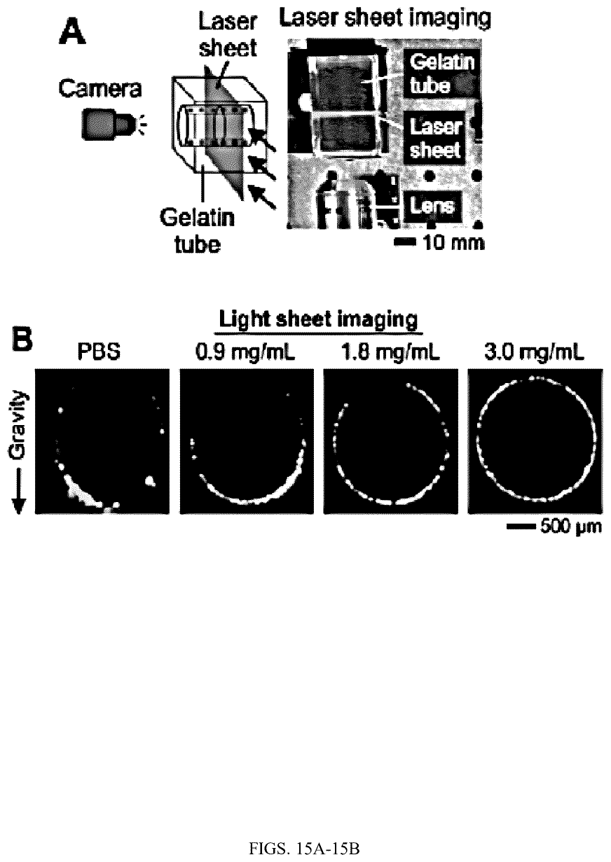

Visualization of particles using a custom-built laser light sheet microscope (LSM): To visualize distribution of fluorescently labeled particles within a transparent material, we constructed an LSM system (5) (FIG. 15) in which a thin light sheet (thickness: ˜5 μm) is created using a 488 nm laser (MDL-D-488, Opto Engine) and cylindrical lenses (ACY254-050-A, Thorlabs). We visualized 10-μm microparticles (excitation/emission: 488 nm/515 nm; Invitrogen) that were deposited onto the inner surface of a tubular hydrogel channel (FIG. 15A). We investigated the effect of the viscosity of delivery medium on the spatial distribution of the seeded particles. The particles were suspended in collagen solutions of different concentrations (0.9-3 mg/mL; 5×10⁶ particle/mL) before instillation into the channel. As the collagen concentration increased, particle distribution became more uniform.

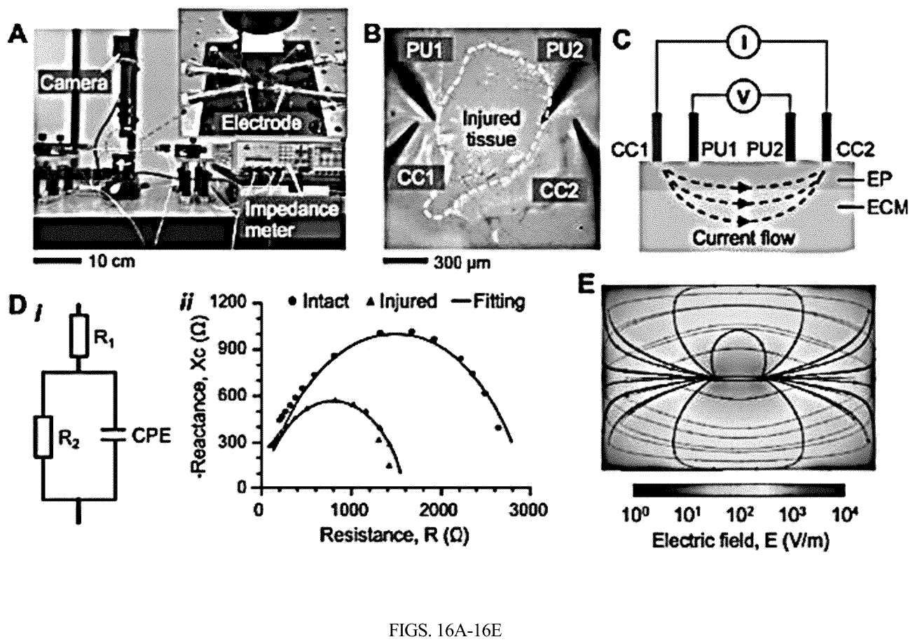

Quantification of airway epithelium integrity via bioimpedance measurement: To investigate the biophysical integrity of the airway epithelium nondestructively (FIG. 16), we created a platform that allowed quantification of the bioelectrical impedance (bioimpedance) of the airway tissue (FIG. 16A-B). This platform utilizes four electrode probes to measure the electrical properties of the tissue (FIG. 16C). During measurements, alternating electrical current (AC) at various frequencies (20 Hz-135 kHz) and 100 μA of magnitude were injected into the tissue using two current carrying electrodes (CC1 and CC2), while two voltage pick-up electrodes (PU1 and PU2) measured the potential distribution (FIG. 16C). The data were analyzed using the Cole model that describes the biological tissue as a combination of a resistor (R) and a constant phase element (CPE) (FIG. 16D, i). The resistance measured at DC (R₀) and infinite AC (R_{∞}), and time constant (τ) for the injured tissue were smaller than those for native tissue, due to impaired epithelium (FIG. 16D, ii). Further, we created a computational model of the epithelial injury that can predict disruption of the tissue bioimpedance (FIG. 16E).

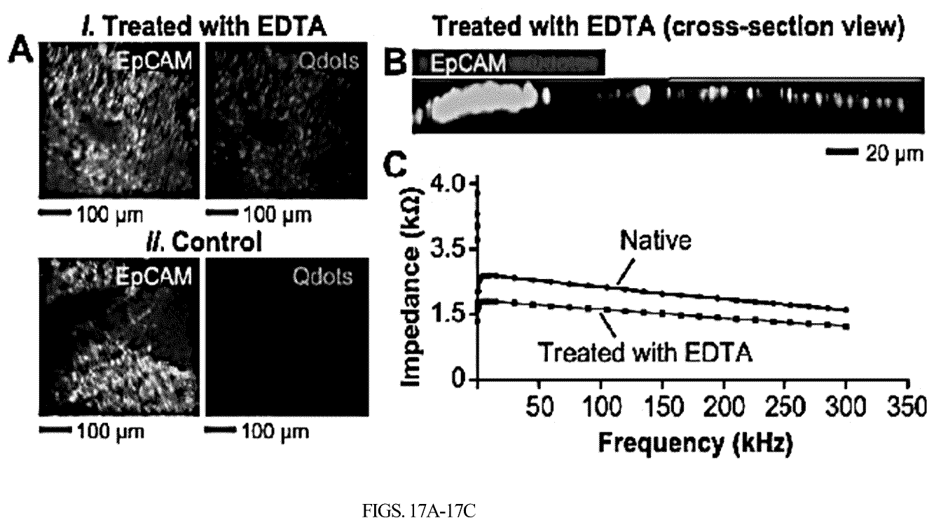

Chemically induced epithelial tight junction disruption: We demonstrated that the tight junctions (TJs) of the airway epithelium can be disrupted by treating the airway tissue with ethylenediaminetetraacetic acid (EDTA) (FIG. 17). In this experiment, the luminal surface of the rat trachea was exposed to 10 mM EDTA for 30 min. While quantum dots (Qdots; emission: 655 nm) could be easily washed away from the native airway tissue (control), Qdots penetrated and remained on the airway tissue surfaces treated with EDTA (FIG. 17A). Notably, Qdots resulted in a weak epithelial cellular adhesion molecular (EpCAM) signal, suggesting disrupted epithelial TJs that increased tissue permeability (FIG. 17B). Further, the EDTA-treated tissue displayed reduced impedance, indicating that the epithelial permeability can be measured in a minimally invasive, real time manner (FIG. 17C).

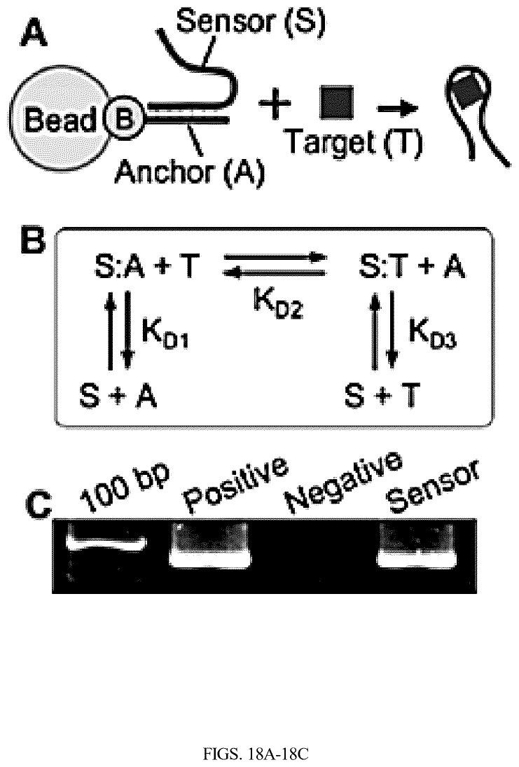

Generation of target-binding nucleic acid probes via competitive binding assay: Our research team has extensive experience in designing and creating nucleic acid-based molecular probes (FIG. 18). For example, Dr. Kim established a microfluidic method that allowed detection, isolation, and enrichment of single DNA strands, known as aptamers, that could bind to specific target molecules or proteins (6, 7) (FIG. 18A). In this aptamer generation protocol, aptamer candidate strands(S) were immobilized onto the surface of streptavidin-coated micro- or nano-beads via biotinylated anchor strands (A). When these beads were exposed to a target molecule (T), the sensor strands that could bind stronger to the target than to the anchor were released from the beads via competitive binding interactions (FIG. 18B). The released target-binding strands were collected, amplified via PCR, and visualized via gel electrophoresis (FIG. 18C). By modifying the sensor strands, such as fluorophore tagging, binding of sensors and target molecules or proteins can be detected and quantified.

Develop bioartificial CF mucus for screening of nanoparticle mobility and mucus penetration: A new biologically relevant and tunable formulation of artificial mucus that matches the physical properties of CF mucus, especially from patients who do not respond to modulator therapy is disclosed. Gene therapy vehicles provided through the Cystic Fibrosis Foundation (CFF) or other entities can be screened for their ability to penetrate biological barriers using the panel of bioartificial mucus formulations we propose to establish.

Model system: Bioartificial mucus formulations will be evaluated and validated in comparison with CF patient sputum samples through an active Institutional Review Board (IRB) approval at Columbia University (IRB-AAAU3193) as a vector mobility screening platform. After validation, the bioartificial mucus will be used to evaluate candidate therapeutic nanoparticles.

Formulation of biophysically accurate artificial CF mucus: To inform the bioartificial mucus formulation, in addition to published data, we will characterize CF patient mucus using in-house material characterization methods, including NMR, IR, HPLC, mass spectrometry, glycoprotein detection kits, and rheometry. Drawing from a large pool of patients will ensure that the bioartificial mucus formulations are representative of the range of mucus properties in CF patients. Notably, nearly one third of the patients being treated at Columbia do not respond to modulator therapy, compared to 10% of CF patients overall. Thus, we will pay particular attention to the mucus obtained from these patients for accurate representation and validation of our model systems.

As noted in the Preliminary Results, early formulations we created already reflect some expected interactions with probe particles but lack increased viscosity and gelation. Thus, rather than focusing strictly on matching biological components until the expected behavior is observed, we will define the minimally required metrics of success for bioartificial mucus that will be used in our proposed studies and as a product deliverable to the CF research community. This product is specifically intended for use in nanoparticle delivery screening and is different from the optimized Pseudomonal growth media reported by others. We identified the user needs, associated metrics, and target specifications for successful creation of bioartificial mucus (Tables 2 and 3). The approach to achieve these values will involve alternative mucin and other protein sourcing; variation of mucin, free DNA, protein, and phospholipid content; and possible inclusion of Pseudomonas biofilm-related components, crosslinking agents, and cell membrane components (e.g., erythrocyte ghosts). In particular, concentration ranges for these components (Table 4, (8-10)) will be refined through material characterization of the mucus obtained from CF patients. Each formulation will be evaluated and benchmarked to the metrics until the target specifications have been reached. Multiple formulations will be developed to reflect a wide range of grades of the patient mucus properties, such as viscosity. Accordingly, multiple target specifications will be set within the measured ranges. Our research team has extensive experience with all methods involved in the preparation of artificial mucus, nanoparticle tracking, preparation and evaluation of samples by electron microscopy and rheology, and other necessary methods.

TABLE 2
User needs for bioartificial mucus.

| | |
|---|---|
| 1 | Particle motion can be meas. by ≥1 technique |
| 2 | Matches micro-rheology |
| 3 | Scalable to hundreds of mL |
| 4 | Widely available materials |
| 5 | Low cost |
| 6 | High specificity screening platform |
| 7 | Biologically relevant pH |
| 8 | Biologically relevant salt concentrations |
| 9 | Matches macro-rheology |
| 10 | Biologically relevant composition |
| 11 | Low biohazard risk |

TABLE 3
Metrics and target specifications for bioartificial mucus.

| Metric | Need | | Importance | | Value | |
|---|---|---|---|---|---|---|
| No. | No.'s | Metric | (1 = high) | Unit | range | Ref. |
| 1 | 1 | Yes/no particle motion | 1 | Y/N | Y | N/A |
| | | measurable | | | | |
| 2 | 2 | Standard probes MSDTS | 1 | μm² | * | N/A |
| 3 | 2 | Standard probes | 1 | μm²/s | * | N/A |
| | | diffusion coefficients | | | | |
| 4 | 3, 4 | Time to produce 500 mL | 2 | days | 10 | N/A |
| 5 | 3, 4, 5 | Cost to produce 500 mL | 2 | $ | ≤500 | N/A |
| 6 | 6 | Specificity | 1 | % | 90-99 | N/A |
| 7 | 7 | pH | 2 | — | 6-8* | 1 |
| 9 | 8 | Concentrations of salts | 2 | mg/mL | NaCl: 9* | 1 |
| | | | | | KCl: * | |
| | | | | | MgCl₂: * | |
| 10 | 9 | Complex modulus | 3 | Pa | ~7.3 + 2.2i* | 9 |
| 11 | 9 | Shear viscosity | 3 | Pa*s | ~51* | 9 |
| 12 | 10 | Number of nonbiological | 3 | — | ≤3 | N/A |
| | | components | | | | |
| 13 | 3, 4, 11 | Biosafety Level number | 1 | — | 1 | N/A |

TABLE 4
Component sourcing and concentration ranges for bioartificial mucus.

| | | | | | Biofilm | Crosslinking |
|---|---|---|---|---|---|---|
| Component | DNA | Mucin | Other protein | Lipid content | components | agents |
| Concentration | 0.16-5.60* | 7.9-15.9** | 0-65.1* | 25.3-41.7* | <2 wt. % ea. | Variable, |
| range (mg/mL) | | | | | [10] | based on agent |
| Potential | Salmon | Porcine | Fetal bovine | Egg yolk (crude/ | Alginate, | EDC + NHS; |
| sources | testes | gastric mucus | serum (FBS); | purified), | Rhamnolipids | periodate/norbornene/ |
| | (commercial) | (commercial, | bovine serum | Cells/cellular | (commercial) | tetrazine [8] |
| | | in-house) | albumin (BSA) | components | | glutaraldehyde; genipin; |
| | | | | | | transglutaminase |

Screening of nanoparticle libraries: Provided with candidate therapeutic nanoparticles through CFF, we will use the bioartificial mucus in conjunction with our particle tracking methodologies to screen nanoparticle mobility in a panel of bioartificial mucus compositions reflecting the distribution of patient mucus viscosities. In an initial high-throughput screening step, each nanoparticle preparation will be tracked in bioartificial mucus via confocal microscopy (for individual particles) and light sheet microscopy (for bulk migration, via fluorescent labeling). In addition, the particles will be screened for penetration through mucus in a Transwell migration assay via Nanosight concentration measurements and stratified by Zetasizer potential measurements (if fluorescent labeling is not possible). Specifically, when fluorescent labeling is possible, quantifiable real-time images of particle distribution in mucus layer cross sections will be acquired via light sheet images. The outputs of this first imaging-based screening step will be root mean square displacement as a function of timescale \sqrt{MSD(t)} that can be directly calculated by correlating the particle position and traveling time and is equivalent to the characteristic diffusion length L_{D} of the nanoparticles. Using this information, we will determine the diffusion coefficient D of the particles using L_{D}=\sqrt{4Dt} for time t and two-dimensional particle tracking. By analyzing the Gaussian distribution of particle displacement, we will calculate the fraction ƒ of nanoparticles that have migrated across a minimum pathlength L (typical mucus layer thickness in CF airways) over a given time period t_{clear} (the timescale of mucus clearance by ciliary action in CF lungs). These data will provide an estimate of the timescale and fraction of vectors expected to reach the mucosal surface when delivered to mucus-covered lungs on cross-circulation in Aims 2 and 3. Particles with \sqrt{MSD(t_{clear})}<<L and low bulk movement (confirmed by light sheet microscopy) will be considered poor candidates for gene delivery. Further, diffusion coefficient D can be used to estimate the time needed for the particles to migrate within mucus. The fraction ƒ of particles that have migrated will be combined with endosomal release data obtained in Aim 1.2 to predict the amount of nanoparticles (i.e., dosing) that should be administered into the lung.

If fluorescent labeling of the nanoparticles is not possible, mucus samples will be placed in the upper compartment of a Transwell, and nanoparticles will be delivered to the top of the mucus sample. Solvent in the lower compartment will be collected at specific timepoints and nanoparticle content collected will be measured by Nanosight and Zetasizer. The expected readouts include: i) nanoparticle flux

$$φ = \frac{particles}{c {m}^{2} * min}$$

and associated time tₚₐₛₛ to traverse a mucus layer of fixed thickness which will also inform dosing, and ii) distribution of particle size and net charge to monitor for aggregation or surface adsorption. Particles with very low flux over 6-12 hours will be considered poor gene delivery candidates because they will most likely fail to reach the airway surface.

Collectively, these first screening steps will also provide insight into which mucus compositions may preclude nanoparticles from efficient transport. In addition to studying which particles are best suited to transport in mucus, we can also study which mucus is best suited to allow particle transport.

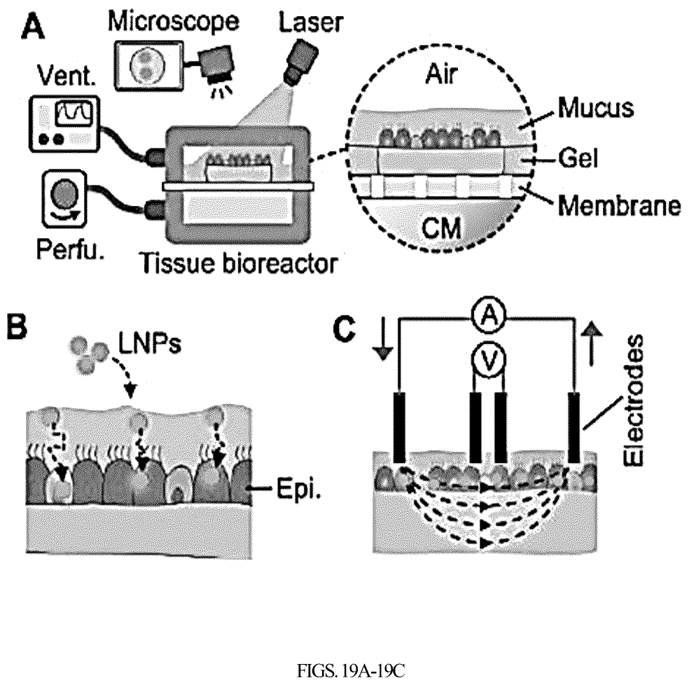

Create a CF-mimetic airway tissue model for in vitro assessment of cellular uptake, endosomal escape, and gene expression. An opto-electrically integrated biorcactor platform is disclosed and a CF-mimetic in vitro airway model with adjustable biophysical characteristics to resemble the environment within the human CF airways, such as mucus thickness, viscosity, and hydration (FIG. 19). This CF-mimetic airway model will be used to investigate how the mucus formulation and the dose and properties of nanoparticles influence internalization and endosomal escape of the cargo. We will then investigate whether transient disruption of the mucosal-epithelial barrier can increase the amount and speed of nanoparticles entering the airway BSCs. To this end, we will use airway mucosal tissue slices obtained from different anatomic regions (e.g., trachea, bronchi, bronchioles) of the porcine and human lungs and deposit bioartificial mucus onto the luminal surface of the airway using our established method (11, 12). When available, we will use airway mucosal tissue slices obtained from CF piglets to validate our CF-mimetic airway tissue model. To achieve real-time and non-invasive tracking and quantification of nanoparticles within the mucus and cells, we will use fluorescent molecular probes and bioelectrical impedance technology.

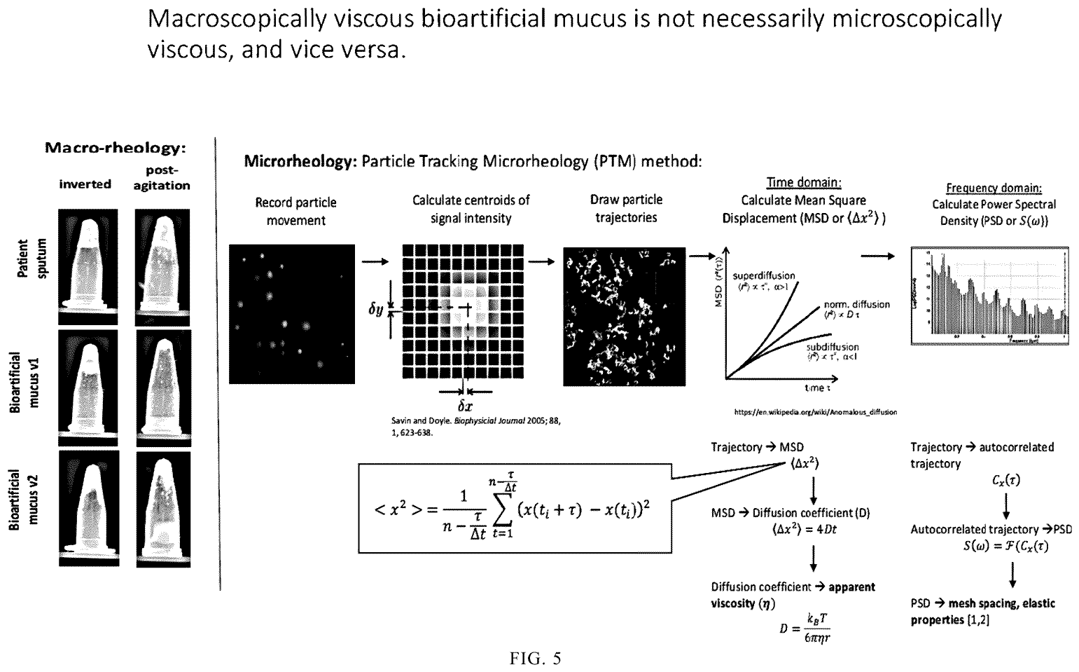

Construction of airway tissue culture platform: We will create a flow-controlled airway tissue bioreactor, coupled with our customized optical and electrical measurement systems (FIG. 19), including the light-sheet microscope (FIG. 5) and bioimpedance analyzer (FIG. 16). The bioreactor chamber will be manufactured using biocompatible and autoclavable Teflon® PTFE, polycarbonate, or other plastics via computer numerical control (CNC) machining (Mini-Mill 3, Minitech®) and laser cutting (P2012+45W, Full Spectrum Laser). Various components of the bioreactor, including the lid and removable imaging inserts for the lids, may be made from polycarbonate by milling, or by laser cutting, or by stercolithography (SLA) printing from resin, or by fused deposition modeling (FDM) printing from PLA filament, or by selective laser sintering (SLS) of nylon powder, or by other 3D printing methods. The culture chamber will consist of 6-20 flow-controlled channels connected in parallel, each of which will be loaded with an airway tissue slice. The channel network will consist of upper and lower compartments (non-limiting example: 6 mm high×7 mm wide×2 cm long inner compartment, with a central 5 mm diameter hole connecting the upper and lower chambers), into which the airflow and perfusate will be introduced, respectively. The two compartments will be separated by a porous polycarbonate membrane bridging the central connecting hole (pore size: 0.4 μm; Nunc™ Cell Culture Inserts, Thermo Scientific™), onto which the tissue slices will be placed with their luminal surface facing the airspace. The upper (airspace) side of the membrane can be coated with 4% (w/v) alginate gel or 2% collagen gel or other biocompatible hydrogeljust before the tissue loading to secure the tissue slices within the channel and prevent leakage of perfusate into the airflow channel. This arrangement will allow tight control of physio-mimetic air-liquid interface at the airway lumen that is important to promote tissue viability. Multi-way connectors (Masterflex Fitting, Cole-Parmer) will be attached to the inlets and outlets of both airflow and perfusion channels via polymer tubing (Tygon® tubing, Cole-Parmer). Airflow will be controlled via a small animal ventilator (PhysioSuite, Kent Scientific), and perfusion flow will be provided via a peristaltic pump (L/S® digital pump system, Cole-Parmer), or both airflow and perfusion may be provided by peristaltic micropumps, such as Bartels mp6 micropumps.

Creation of CF-mimetic airway tissues: Using this platform, we will create CF-mimetic airway tissues from mucosa obtained from freshly explanted lungs of euthanized Yorkshire swine (weight: ˜40 kg; n=10 animals) and human donor lungs (from lung transplant programs) (FIG. 19A). While porcine tissues will be used for most studies, human donor lungs will be used to replicate a subset of studies, and the results will be compared. We will also obtain mucosa from explanted lungs of CF piglets when available to compare with findings in non-CF swine and human lungs. Airway tissues will be harvested from different regions of the lung (e.g., upper, mid, or lower airways) and sectioned. Disk-like tissue slices (diameter: 1-5 mm, thickness: 0.5-2 mm) will be gently washed in PBS buffer and transferred into the bioreactor. As described above, alginate gel or collagen gel will be coated onto the porous membrane prior to the placement of the tissue slice, and the lid of the tissue culture chamber will be closed. After the gel is solidified (≤5 min), the perfusion channel will be filled with cell culture medium (e.g., Airway Epithelial Cell Culture Medium, Lonza; or Dulbecco's Modified Eagle's Medium; or other) and the airflow channel will be ventilated (breathing rate: 10-20 bpm; tidal volume: matched to flow parameters in upper, mid, and lower airways). We will instill a small bolus of mucus (volume dependent on mucus viscosity and capillary number) through each channel containing the airway slice to create a mucus layer via our previously established plug flow film generation technique (12). The device will be ventilated with ambient air for 5 min to improve mucus adhesion onto the airway surface. To create thicker mucus film (>100 μm), the bolus instillation procedure will be repeated. The correlation between mucus delivery and thickness will be evaluated via microscopy of the airway cross-section following the mucus deposition. Viscoelastic physical properties of the deposited mucus will be measured by our custom-built vacuum-based rheometer directly on the airway surface.

Real-time tracking of nanoparticle transport: The CF-mimetic airway tissues will be used to correlate the dose of nanoparticles delivered onto the mucus with the amount and speed of particle internalization (FIG. 19B,C). Specifically, CF-mimetic airways will be coated with different thicknesses of mucus film (0.1-2 mm) to establish a mucus barrier to particle transport. Nanoparticles will be administered via nebulization or instillation. To modulate the particle density deposited onto the mucus surface, we will use different concentrations of nanoparticle solutions (1-200 mg/mL), while all other delivery conditions (exposure duration, instillation speed, liquid volume) will remain the same across experimental groups. Following nanoparticle delivery, their transport will be monitored directly, continuously, and non-destructively within the bioreactor using our opto-electrically enabled particle tracking method: the nanoparticles will be traced using a light-sheet microscope by labeling the particles with lipophilic membrane dye (e.g., BioTracker NIR770, Sigma Aldrich).

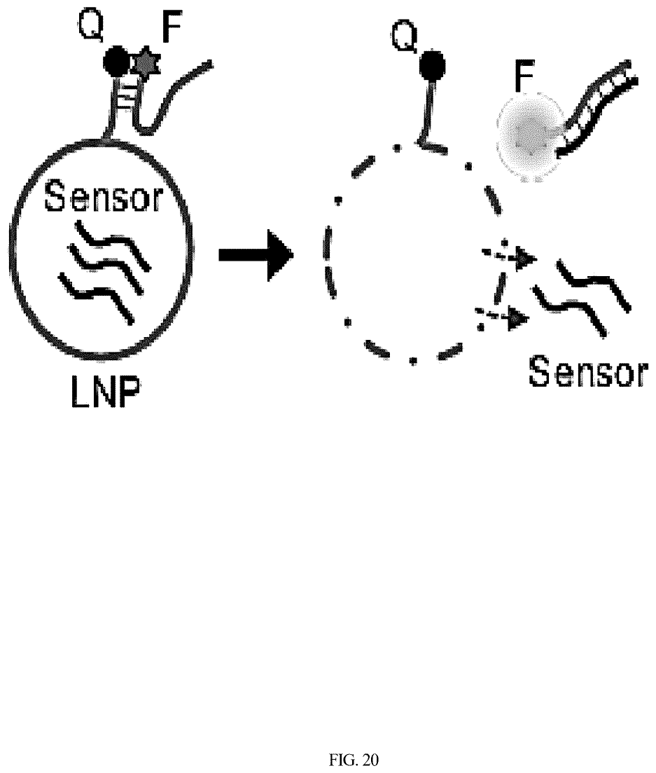

To monitor optically the endosomal escape and release of gene or mRNA into the cytoplasm, nanoparticles will be further incorporated with FRET- or quenching-enabled molecular beacons (FIG. 20). For example, electrically neutral peptide nucleic acid (PNA) anchor strands, of which one end is labeled with a quencher (e.g., BHQ-3, PNA Bio Inc.), can be tethered to the nanoparticles. PNA reporter strands that can partially hybridize to the anchor strands will be designed with the hybridizing end labeled with a fluorophore (e.g., Cy5, PNA Bio Inc.). The remaining region of the reporter strands will be created to be complementary to PNA-based sensor strands encapsulated into nanoparticles. This will allow the sensor strands released from nanoparticles following endosomal escape to hybridize to the reporter strands, leading to detachment of the reporter strands from the anchor (quencher) strands and subsequent emission of fluorescent signals. In parallel, we will quantify endosomal release of the gene by measuring the electrical properties of the airway tissue using our bioimpedance measurement method (FIGS. 16 and 17). We expect that the release of negatively charged gene or mRNA from nanoparticles into the cytoplasm will result in measurable changes in the bioimpedance of the epithelial cell layer. These studies will be completed in both porcine and human tissues, towards validating the porcine tissue as a model of human tissue, and to identify any shortcomings of the porcine model.

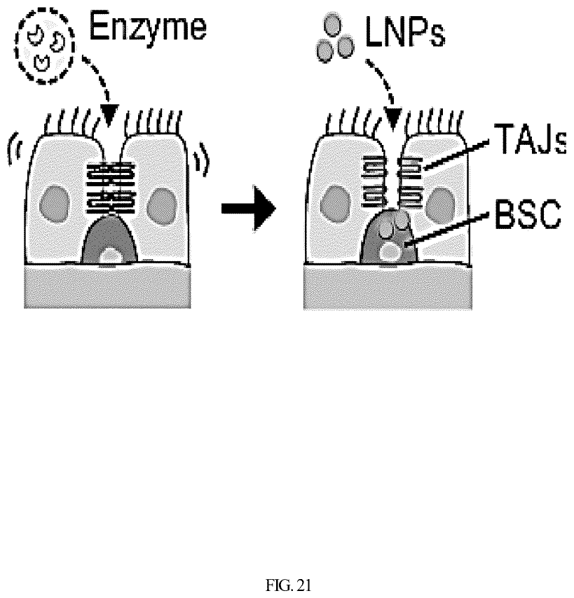

Particle delivery to BSCs via transient epithelial barrier disruption: Whether particle delivery, internalization, and release into BSCs can be promoted by pre-conditioning the airway surface by mechanically assisted clearance of mucus, followed by transient disruption of the tight/adherens junctions (TAJs) and reduction of ciliary motion (FIG. 21), can be determined using CF-mimetic airway tissues from porcine and human lungs. TAJ disruption will be confirmed by reduced bioimpedance of the airway epithelium, and the ciliary motion (beating frequency, stroke) will be measured optically. To improve real-time visualization of the cilia, ciliated cells will be labeled fluorescently with FITC-WGA (Vector Laboratories; Excitation/Emission: 488 nm/525 nm) prior to the experiments using our established protocol. To promote mucus removal, the CF-mimetic tissues will be exposed to mechanical oscillation (frequency: 20 Hz; amplitude: 5 mm; duration: 1 min) and irrigated with hypertonic saline (100 μL of 7% NaCl), as in our preliminary studies (FIG. 13). Removal of the mucus layer will be monitored using our custom-built light-sheet microscope coupled to the tissue chamber.

To disrupt the TAJs and reduce the cilia motion simultaneously, the airway lumen will be coated with ethylenediaminetetraacetic acid (EDTA) methylcellulose solution. Following 30 min of incubation, disruption of TAJs will be monitored via bioimpedance measurement, while cilia beat frequency and stroke distance will be monitored under a fluorescent microscope. Following the confirmation of the TAJ and cilia beat disruption, we will introduce the fluorescently labeled nanoparticles via nebulization or instillation. Nanoparticle transport across mucus and through the gaps between epithelial cells will be monitored and quantified via confocal microscopy (FIG. 17), light-sheet microscopy (FIG. 15), immunohistochemistry, and PCR. These studies will be completed in both porcine and human tissues for model validation.

Comparative evaluation of nanotherapeutic delivery and therapeutic effectiveness beyond the duration of ex vivo support: Successful gene therapy for CF requires effective uptake and sustained expression of CFTR cargo. Since ex vivo support of whole lungs is limited in duration to ˜100 hours (13), longer-term studies would be needed to assess sustained therapeutic efficacy. Therefore, we will obtain porcine and human lung tissue specimens (e.g., trachea, bronchi, airways) at the endpoint of cross-circulation, and support these tissues for long-term analysis with the in vitro bioreactor developed herein. We will use the opto-electrically integrated tissue culture platform for comparative longitudinal studies of gene delivery, uptake, endosomal release, and gene expression in both human and porcine lung tissues for up to 2 weeks. As it is unknown how the porcine tissues will respond to the gene therapy, these studies constitute an important step towards validating the ex vivo porcine CF-mimetic lung model as a reliable surrogate for CF-mimetic human lungs.

Model system: We will obtain biopsies of porcine and human lung tissue following termination of ex vivo support (EVLP or cross-circulation) that will allow for studies of gene therapy efficacy beyond the duration of ex vivo support. The opto-electrically integrated mucosal tissue bioreactor generated herein will be used with porcine and human tissue biopsies after therapeutic intervention for long-term monitoring up to 2 weeks.

Longitudinal studies of ex-vivo treated tissues: Longitudinal analysis of therapeutic efficacy will be performed following cross-circulation by combining porcine and human lung biopsies (obtained at cross-circulation endpoint) with the mucosal tissue bioreactor developed above. We will assess therapeutic efficacy including gene expression and physiologic changes for a period of up to 2 weeks, in order to quantify long-term efficacy and/or determine a need for redosing. Specifically, we will monitor sustained expression of CFTR and the luciferase reporter gene with PCR and bioluminescent imaging. To monitor physiologic changes resulting from correction of CFTR, we will utilize bioimpedance measurements, mucus viscosity, pH, and composition. A successful nanotherapeutic regimen will lead to sustained CFTR expression with corresponding changes in airway physiology, including improved mucus viscosity and barrier function.

Expected Outcomes: The objective is to generate biophysically accurate artificial CF mucus, integrate it with the opto-electrically enabled bioreactor, and use this high-throughput system to screen libraries of nanoparticles for CFTR gene therapy. Specific outcomes will include (i) bioartificial mucus product that can be distributed to the CF community, (ii) transport kinetics data (\sqrt{MSD(t)}, D, f, φ, tₚₐₛₛ) for nanoparticles with different properties, (iii) timescale of delivery and dosing estimations, (iv) optimized opto-electric system and reference measurements, (v) comparison data of delivery to the pretreated tissue surface (to disrupt tight junctions) vs. unaltered model CF tissue, (vi) correlation of nanoparticle delivery kinetics and uptake in porcine and human tissues, and (vii) extended gene expression data (and interspecies comparison) for porcine and human tissues following their treatment on EVLP or cross-circulation. PNA strands can be used, for example, as the fluorescent molecular sensors. Because PNA is an electrically non-charged molecule, it is expected that decoration of the nanoparticle surface with PNA will not alter the electrical properties of the nanoparticles.

Development of CF-mimetic porcine lung platform for evaluation of nanotherapeutic delivery, cellular uptake, and expression: Nanoparticles containing genetic cargo administered into the respiratory tract must overcome several barriers to effectively reach appropriate cell types and correct CFTR in situ: (i) delivery to the appropriate regions of the lung, (ii) penetration through highly viscous CF airway mucus, (iii) uptake by appropriate cell types, (iv) endosomal escape, (v) gene expression in relevant cell types, and (vi) functional changes resulting from expression of CFTR in relevant cell types.

In developing an ex vivo model of the CF airway using whole porcine lungs with bioartificial mucus and to apply this model can evaluate the delivery of nanotherapeutics. This model system will incorporate bioartificial mucus and establish real-time methods for assessing delivery efficacy, cellular uptake, and expression. Studies in this aim will result in: (i) development of a porcine lung model that mimics the CF airway environment, definition of baseline functional parameters for dissemination of the model to the CF research community, (iii) determination of optimal modalities for nanotherapeutic delivery using CF-mimetic lung model, and (iv) evaluation of CF-mimetic lung responses to therapeutic deliveries.

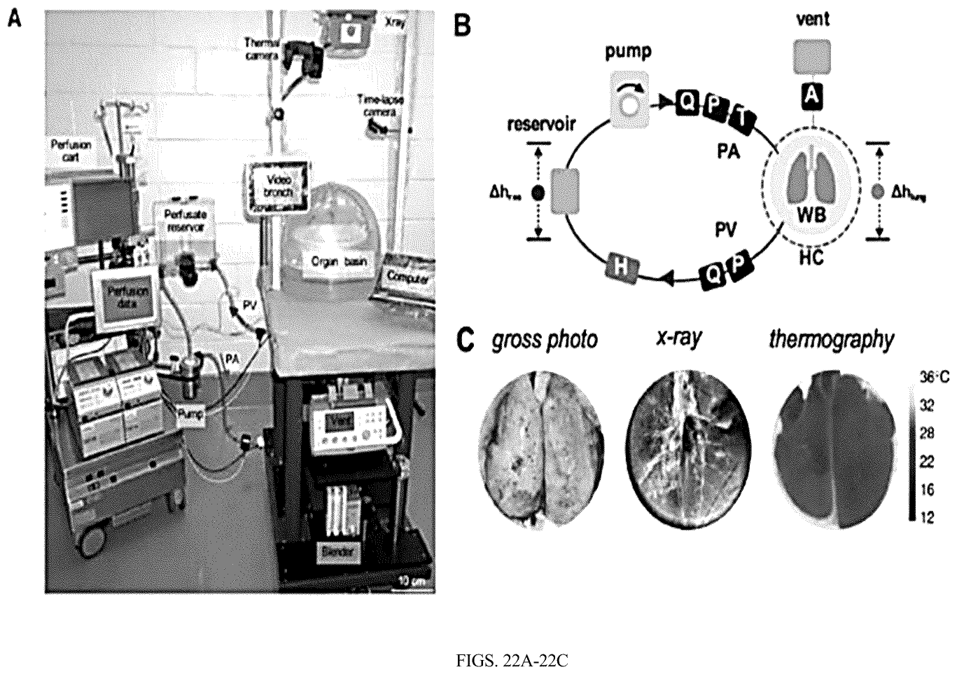

Preliminary results: Ex vivo lung perfusion platform: We established an ex vivo lung perfusion (EVLP) platform with normothermic perfusion to support the CF-mimetic porcine lung for real-time evaluation of nanoparticle delivery methods and distribution. The platform enables monitoring of lung function and real-time fluorescent lung imaging for localization and quantification of delivered therapeutics. The platform also provides assessments of the whole lung via oximetry, radiography, thermography, and microscopy (FIG. 22).

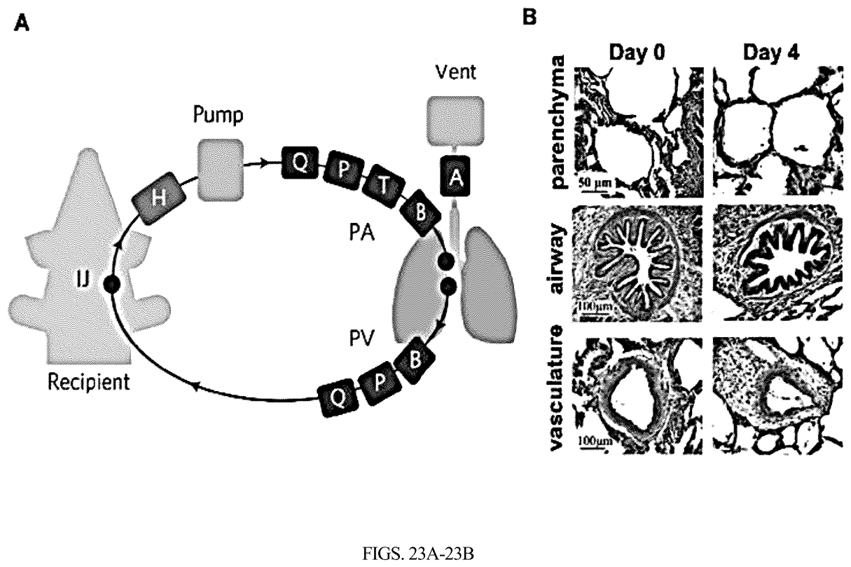

Cross-circulation platform: Because EVLP is limited to organ support times of <12 hours, we developed a cross-circulation platform for long-term support of ex vivo porcine or human lungs. This innovative cross-circulation platform is integrated with computerized sensors and imaging devices that enable support of ex vivo lungs for multiple days, measurement of lung function in real-time, and delivery of therapeutic cargo directly to the lung airways and vasculature. Notably, our porcine cross-circulation platform supported lungs for up to 4 days with outstanding structural and functional maintenance of lung parenchyma, airways, and vasculature (FIG. 23). Cross-circulation also allowed real-time assessment of lung function and localized therapeutic interventions, including alveolar recruitment, bronchoalveolar lavage, surfactant therapy, and cell and nanoparticle delivery. Multi-modal on-line imaging and analytic capabilities enabled by cross-circulation also include bronchoscopy, radiography, thermography, metabolic assays, and the direct measurements of oxygenation and compliance.

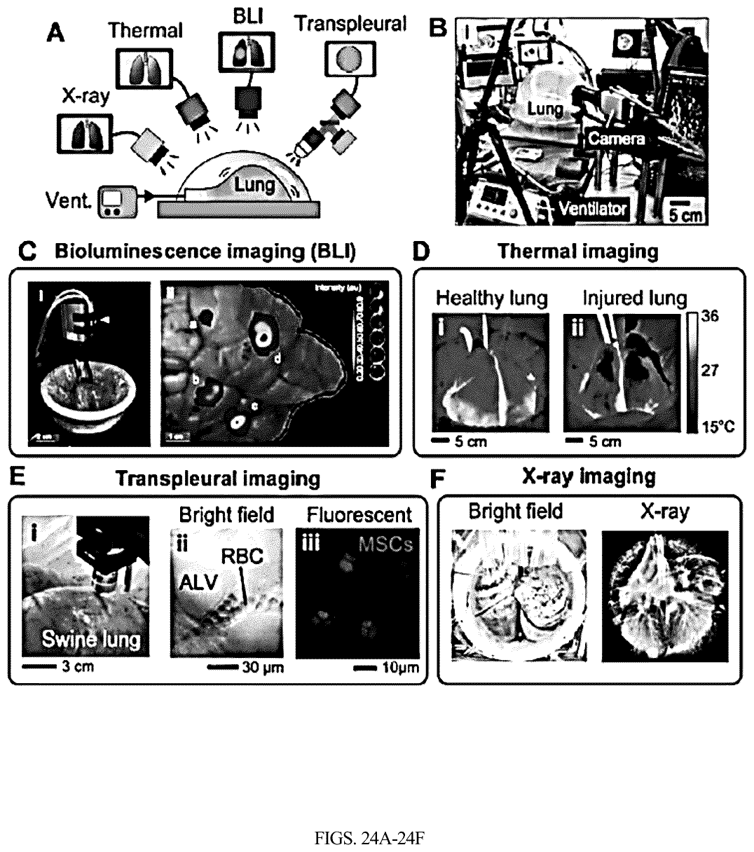

Integration of non-invasive imaging: We integrated different imaging modalities into our cross-circulation platform to non-invasively monitor the status of the lung during ex vivo support (FIG. 24A,B). In particular, we created a bioluminescence imaging system compatible with both the EVLP and cross-circulation for localization and quantification of bioluminescent tagged therapeutics (FIG. 24C). A thermal imaging modality allowed us to continuously monitor the temperature of the lung that was correlated with the blood perfusion of the lung (FIG. 24D). Transpleural imaging enabled non-invasive microscopic visualization of the inner space of the lung, such as alveoli and capillary blood vessels. We visualized fluorescently labeled cells seeded in the alveoli using this technique (FIG. 24E). A portable X-ray scanner (PXP-16HF, United Radiology Systems) was coupled to the platform to monitor the airspace recruitment during the ex vivo support of the lung (FIG. 24F).

Liquid film deposition in a tube via liquid plug instillation: We investigated the underlying mechanism of the liquid film generation by liquid plug instillation using a tubular hydrogel channel (11, 12) (FIG. 15A). When a liquid plug is introduced through a channel with a wettable surface, a thin layer of liquid film is deposited at the rear end of the plug. The thickness of the liquid film (h) was proportional to the capillary number of the plug (Ca=μU/σ) as h˜ Ca^{2/3}, where μ is liquid viscosity, U is plug speed, and o is liquid surface tension (14, 15) (FIG. 15B). We experimentally verified this finding by investigating the movement of a small volume (0.5-10 μL) of PBS solution infused through a hydrogel channel (FIG. 15C). The channel length wetted by the plug was directly proportional to the initial plug volume (V₀) (FIG. 15D). Additionally, we determined the relationship between the film thickness (h) and plug capillary number (Ca) as h=1.6Ca^{2/3} (FIG. 15E). The findings indicate that the flow regimes of the artificial mucus would dictate the coverage and thickness of mucus film in the airway.

Liquid film generation in the rat airway: To establish liquid film generation on the surface of the rat airway (12), we constructed a near-infrared (NIR) imaging system combined with a ventilator, with which small liquid plugs (volume: 35-110 μL) infused into the lungs through the trachea were monitored externally (FIG. 16A). The plugs were added with indocyanine green (ICG) molecules (excitation: 785 nm; emission: 820 nm) to visualize their movement and film generation within the lung (FIG. 16B, i-iii). A mathematical model was formulated that predicted liquid film generation in different anatomical lung regions (FIG. 16C,D). We demonstrated deposition of liquid film into predefined respiratory regions. We used liquid plugs containing carboxyfluorescein diacetate succinimidyl ester (CFSE), which fluorescently labeled the airway epithelium in contact with the liquid film. Fluorescent images of lung cross-sections showed that when 35 μL of CFSE solution was instilled, fluorescent signal indicating film deposition was observed only in the large airways (G₀-G₅) (FIG. 17A). In contrast, fluorescence was detected throughout the proximal airways (G₀-G₁₅) of the lungs instilled with 110 μL of CFSE. In addition, some peri-bronchiolar alveoli surrounding the target airways showed fluorescence due to diffusion of CFSE from the target airways (FIG. 17B).

Non-destructive measurements of mechanical tissue properties: We developed a vacuum-based method that can allow non-destructive measurement of mechanical properties of lung tissues (16) (FIG. 18A, i). In this approach, vacuum pressure (P_{V}) is applied directly to lung tissue via a tubular probe to gently elongate local tissue to a certain length (L_{E}) (FIG. 18A, ii). Results showed that tissue elongation was inversely related to the alveolar pressure (P_{Alv}). As the lung volume increases with PAN, the lung tissue becomes stress-bearing, leading to decreased L_{E}(17, 18). As P_{Alv} increased from 2 to 10 cm H₂O, L_{E} decreased from 0.33±0.05 to 0.12±0.03 mm when −2 kPa of P_{V} was applied (FIG. 18B). Further, L_{E} of rat lung tissue varied nonlinearly in response to P_{V} while the loading-unloading curves showed hysteresis which is a feature of lung tissue (FIG. 18C). As pressure inside the lung increased, L_{E} decreased. For PAN of 2, 5, and 10 cm H₂O, L_{E} were 0.33±0.05, 0.20±0.03, and 0.12±0.03 mm (FIG. 18D). By correlating P_{V} and L_{E}, clastic moduli (E) were determined via E=3 kR_{P}P_{V}/2πL_{E}, where k is a constant specific to the probe geometry and R_{P} is the probe radius (19-22). For 2, 5, and 10 cm H₂O of P_{Alv}, E were 4.4±0.6, 7.4±1.1, and 13.2±3.9 kPa, respectively (FIG. 18E).

Generate a CF-mimetic porcine lung ex vivo utilizing bioartificial CF mucus: Delivery of bioartificial mucus to ex-vivo porcine lung: Evaluation and validation of CF-mimetic mucus delivery and dosage will be determined utilizing ventilated lungs of Yorkshire swine (weight: ˜40 kg; n=12 animals). A CF-mimetic mucus plug will be introduced into the ventilated porcine trachea using our established protocol (23, 24). Initial mucus plug volume and ventilation conditions will be informed by mathematical modeling, as in our previous studies (12). The lungs will be on positive-pressure ventilation, for pressure-induced advancement of the mucus plug. A mucus film of a specified thickness will be established by setting the plug volume and airflow rate (12) in our custom micro-catheter delivery system (25). We will evaluate mucus delivery in both prone and supine positions, and ventilate the lung for 10 min to allow mucus distribution.

Validation of mucus delivery: Several different strategies will be employed to confirm delivery of CF-mimetic mucus to the ventilated porcine lung. We will visualize mucus distribution by bronchoscopic imaging, and confirm it in real-time by adding ICG fluorescent dye. We will use our transpleural near-infrared imaging system (FIGS. 24 and 16) to visualize the distribution of mucus-ICG solution across the lung airspace (12). Mucus deposition in porcine airways will be confirmed by histology (26), following addition of CFSE which will fluorescently label viable airway epithelium that came into contact with mucus (12).

Ciliary mucus clearance: Mucociliary clearance mechanisms in healthy porcine lungs may remove exogeneous CF-mimetic mucus. Therefore, mucus clearance and re-dosing (volume, frequency) will be assessed in porcine lungs on EVLP. Mucus samples will be taken from different airway generations at different timepoints and analyzed using NMR, IR, HPLC, glycoprotein detection assays, and rheology.

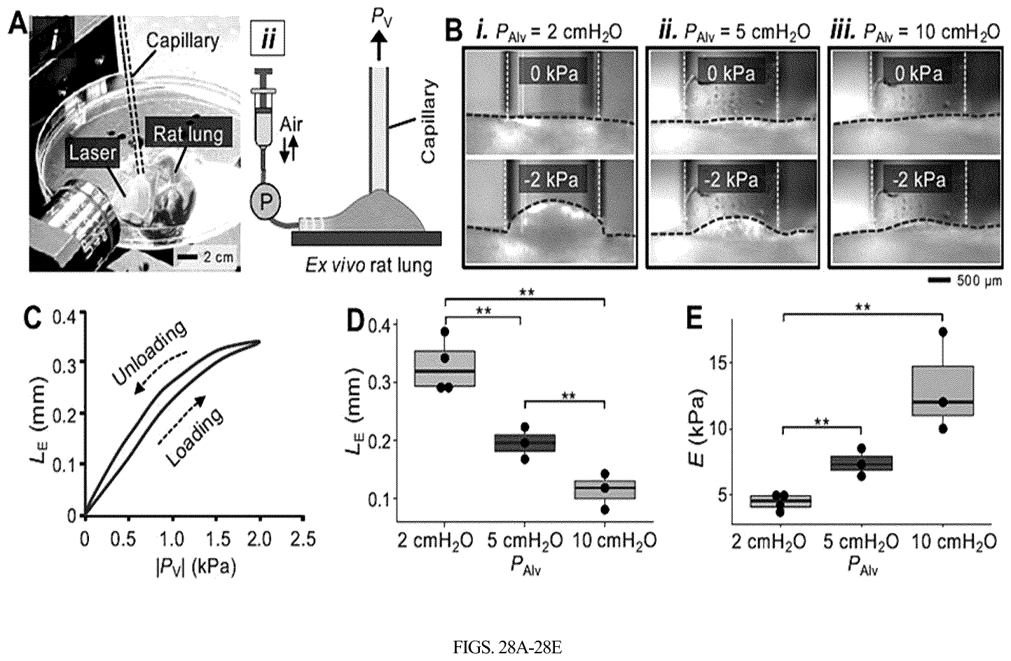

Functional changes in porcine lungs after mucus administration: We will evaluate the mechanical properties and functional changes for the ventilated ex-vivo porcine lungs with CF-mimetic mucus. This will be done using our non-destructive tissue elasticity quantification via vacuum-assisted stiffness measurement (FIG. 28) and whole lung compliance measurements in all anatomic lobes (RUL, RML, RLL, LUL, LLL) (27) (FIGS. 12 and 13). We will compare these data to the same porcine lungs prior to administration of CF-mimetic mucus. In all lungs, we will evaluate pressure/volume loops using our established methods (25).

Investigate optimal nanotherapeutic delivery methods in porcine CF-mimetic lungs on EVLP. We will apply the porcine CF-mimetic mucus developed to investigate nanotherapeutic delivery methods in whole porcine lungs ex vivo. Porcine lungs will be supported on EVLP for short-term studies (<12 hours) that will inform the optimal delivery method and allow real-time evaluation of nanoparticle distribution in the lung.

Model system: Explanted Yorkshire swine lungs (weight: ˜40 kg; n=8 animals) will be supported using EVLP for up to 12 hours, to evaluate distribution patterns of nanoparticles in the airways. Bioartificial mucus will be introduced according to the methods developed above. The EVLP platform will enable in situ monitoring of physiologic parameters including oxygenation and compliance via our custom-built systems incorporated into the platform. To promote perfusion, the lungs will be cannulated using the vascular “bio-bridge” as a conduit from the left atrial cuff as our team successfully demonstrated in our previous studies (24, 25, 28). Lungs will be placed in a preservation chamber (XVIVO) while being supported via computer-controlled ventilator and pumps (29).

Nanoparticle delivery: Using porcine lungs maintained on EVLP, we will study various methods of nanoparticle delivery, including but not limited to: aerosol inhalation (30, 31) and targeted bronchoscopic instillation (32). We will use a commercially available nebulizer (Acroneb, Kent Scientific) that can generate aerosolized droplets ranging in diameter from 2.5-6 μm. It is known that droplets with a diameter of >5 μm tend to be deposited in the upper airways, while droplets <5 μm in diameter tend to be deposited in the lower airways (33). We will therefore examine aerosolized particles of various sizes to optimize their distributions. In parallel, a flexible bronchoscope will be inserted via an endotracheal tube, with a fine catheter inserted through the bronchoscope channel for delivery of small volumes of nanotherapeutic into the segmental bronchi. After bronchoscopic delivery, an inspiratory hold maneuver will be performed to facilitate distribution throughout the lung. The goal of this sub-aim is to determine nanoparticle localization patterns within the porcine airways using various delivery methods. Therefore, these studies may be conducted with nanotherapeutics void of gene therapy cargo in an effort to conserve resources.

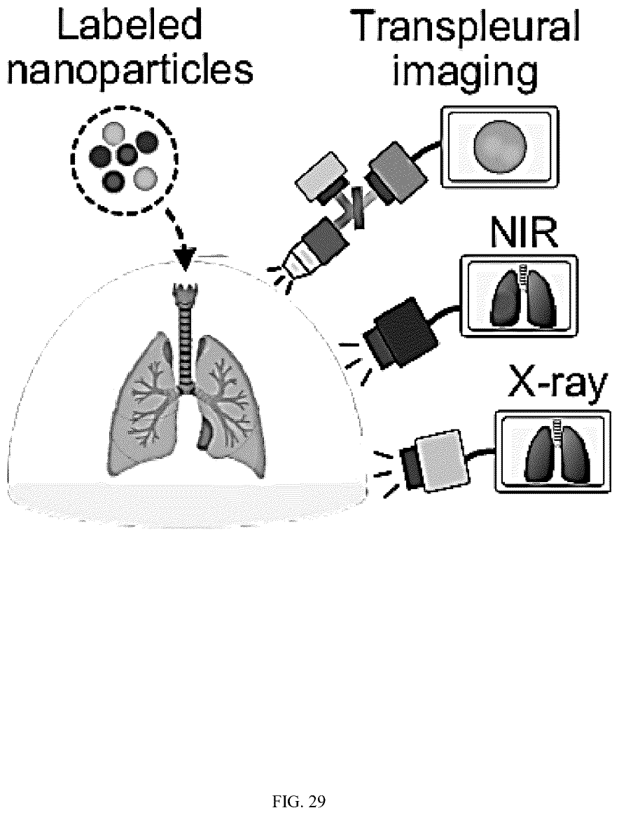

Real time localization of delivered nanoparticles: We will evaluate the distribution of nanotherapeutics within the lung airways using in-situ imaging (FIG. 29). Nanoparticles carrying fluorescent surface makers or cargo will be imaged using a custom-built transpleural fluorescent microscope system, consisting of an LED excitation source, objective, and camera. Nanoparticles with either radiopaque metallic properties or ICG labeling can also be imaged within the lung using X-ray and near-infrared imaging, respectively. Intratracheal administration of aerosolized radiopaque nanoparticles (gold or iron-oxide) followed by X-ray imaging in a murine model (34) can be readily extended to the porcine lung. We will image the lung every hour following therapeutic delivery to determine the kinetics of distribution throughout the airways. The transpleural fluorescent imaging, X-ray, and near-infrared imaging will allow us to determine in real-time the nanoparticle localization patterns within the porcine airway for various methods of delivery. We will also study porcine lungs preconditioned with high frequency oscillation, nebulized hypertonic saline, or dornase alpha. If the label intensity is augmented by airway preconditioning (>20%), this will be employed

Evaluate cellular uptake and expression of nanotherapeutics in porcine CF-mimetic lungs on cross-circulation.

We will develop an ex vivo human-sized model of CF using porcine lungs and bioartificial CF mucus, and use this model to screen nanotherapeutics for treatment of CF. Additionally, we will develop a platform technology for ex vivo support and real-time evaluation of lung function and the quantification of nanotherapeutic distribution and uptake. This platform will be designed to serve as a robust whole lung model of CF for the CF research community.

Model system: Ex vivo porcine lungs (weight: ˜40 kg; n=8 animals) with bioartificial mucus will be supported on cross-circulation for up to 48 hours (or longer if needed) to deliver nanotherapeutics and monitor therapeutic efficacy. Cross-circulation provides systemic support of the lungs in a near-physiologic environment and enables real-time analysis of lung function and vascular health. Cross-circulation also enables multi-modal delivery of therapeutics including intravascular (not studied here), bronchoscopic, and nebulized administration. We will procure Yorkshire porcine lungs and establish cross-circulation with the living porcine host as we previously reported (24, 25, 35). Mucus will be introduced using methods developed above. Lung functional parameters (oxygenation, airway and transepithelial pressures, compliance) will be closely monitored. Radiographic and bronchoscopic images will be obtained to assess distribution of mucus and any airway obstruction. In control experiments, we will administer nebulized saline to the lungs following mucus instillation and monitor lung function to establish the baseline parameters of the model. These baseline parameters will serve as: (i) benchmark for comparison following therapeutic administration, (ii) metrics for cross-validation of the platform across the two research sites (Columbia and Vanderbilt), and (iii) quantitative ‘check-points’ for the broader research community to utilize the platform for therapeutic testing. Using bronchoscopy and bronchoalveolar lavage collection, we will visualize gross changes to the mucus and quantify changes in airway fluid composition. Mucus sampling via bronchoscope will enable longitudinal monitoring of viscosity, stiffness, and composition.

Monitoring cellular uptake and clearance of delivered nanoparticles: Following definition of CF-mimetic model baseline parameters, we will deliver fluorescently-labeled nanotherapeutics with CFTR cargo into the lungs by a bronchoscope or nebulizer. The variables to be tested will be the nanoparticle formulation, dose, and modality of administration. Following therapeutic administration, we will monitor changes to lung functional parameters relative to the baseline values. To monitor penetration and uptake of the nanoparticles we will bronchoscopically collect mucus samples following administration and observe retention of particles within mucus on a fluorescent microscope. A decline in the concentration of particles in mucus over time would indicate either successful uptake or clearance (e.g., entrapment in distal lung or removal via mucociliary clearance). We will distinguish between the uptake and clearance using periodic distal lung biopsies and bronchoscopic mucus sampling from the region most proximal to the endotracheal tube, respectively.

We will monitor the distribution and uptake of nanoparticles throughout lung regions using our custom-built whole lung bioluminescent imaging platform (FIG. 14). Introduction of a luciferase reporter gene will result in bioluminescent signal detectable only in cells that uptake nanoparticles. In comparison, delivered (but not internalized) nanoparticles will emit a fluorescent signal which we will visualize by our fiber-optic fluorescent probe. The combined bioluminescent-fluorescent imaging will determine the relative efficacy of nanoparticle uptake and endosomal escape. If mucus penetration and uptake of nanoparticles are poor (<10%), we will consider pre-treating the lungs with nebulized saline or dornase alpha (Pulmozyme).

Measurement of CFTR gene expression in CF-mimetic porcine lungs following therapeutic delivery: Following confirmation of distribution, mucus penetration, and cellular uptake of nanoparticles, we will investigate expression of the genetic cargo (e.g., CFTR, reporter genes) in the lung. Based on the measured nanoparticle distributions, we will obtain tissue samples from known nanoparticle delivery regions via bronchial biopsy and surgical wedge resection (parenchyma). To determine the timeline of cellular uptake and expression, we will perform serial sampling every 4 hours for longitudinal comparison, and subject them to RNA sequencing and PCR to quantify expression levels of CFTR. We will utilize Western blot and immunofluorescent staining to assess CFTR protein. Because expression of CFTR is also expected in healthy porcine lungs, in addition to longitudinal comparison we will also compare expression levels with those measured in porcine lungs with CF-mimetic mucus treated with only nebulized saline. Mucus samples from the airways will be analyzed by mass spectrometry to determine the changes in mucus composition following gene delivery. We will also measure changes in mucus viscosity and elastic modulus over time. To assess phenotypic and functional changes resulting from gene delivery, we will monitor lung function (oxygenation, airway pressures, compliance). Finally, we will measure the changes in airway bioimpedance following the introduction of CFTR.

Expected Outcomes: The objective is to develop an ex vivo model of the CF airway using whole porcine lungs with bioartificial mucus and to utilize this model to evaluate delivery of nanotherapeutics. The proposed studies will: (i) develop an optimized methodology for introduction of CF-mimetic mucus into the lung airways, (ii) establish nanotherapeutic delivery throughout airway generations, (iii) quantify therapeutic efficacy by measuring cellular uptake and gene expression of delivered nanotherapeutic cargo, and (iv) compare data collected in porcine lungs with those collected with human lungs, to probe and validate the model.

Mucus can be advanced into the more distal lung regions (e.g., small airways), with a miniaturized catheter (made, e.g., via 3D printing or machining) to directly instill mucus. For nanotherapeutic delivery, a commercially available nebulizer is used to generate aerosolized droplets ranging in diameter from 2.5-6 μm (Aeroneb, Kent Scientific). For droplets of 200 nm ofr less in diameter, to reach the most distal conducting airways the aerosolizer is modified to operate at higher pressure and generate smaller droplets. Penetration of the mucus barrier is a key consideration for effective nanotherapeutic uptake. For nanoparticles with poor yield of cellular uptake (<10%), we will facilitate delivery by pre-treating lungs with dornase-alpha. Alternatively, we may employ hypertonic saline to hydrate the mucus layer via osmotic forces to assist with nanoparticle movement. Fixation with paraformaldehyde or freezing can be used for histological visualization of mucus.

Evaluate nanotherapeutic delivery, cellular uptake, and expression in human lungs ex vivo: To screen nanotherapeutic delivery, uptake, and gene expression in human lungs, the model will be used. We aim to verify that the CF-mimetic porcine lung model (Aim 2) is sufficiently similar to human lungs (Aim 3) to serve as an ex-vivo model of cystic fibrosis. Studies in this aim will validate: (i) porcine CF-mimetic lung model as a representative platform for therapeutic testing and (ii) nanoparticle delivery, cellular uptake, and expression in human lungs.

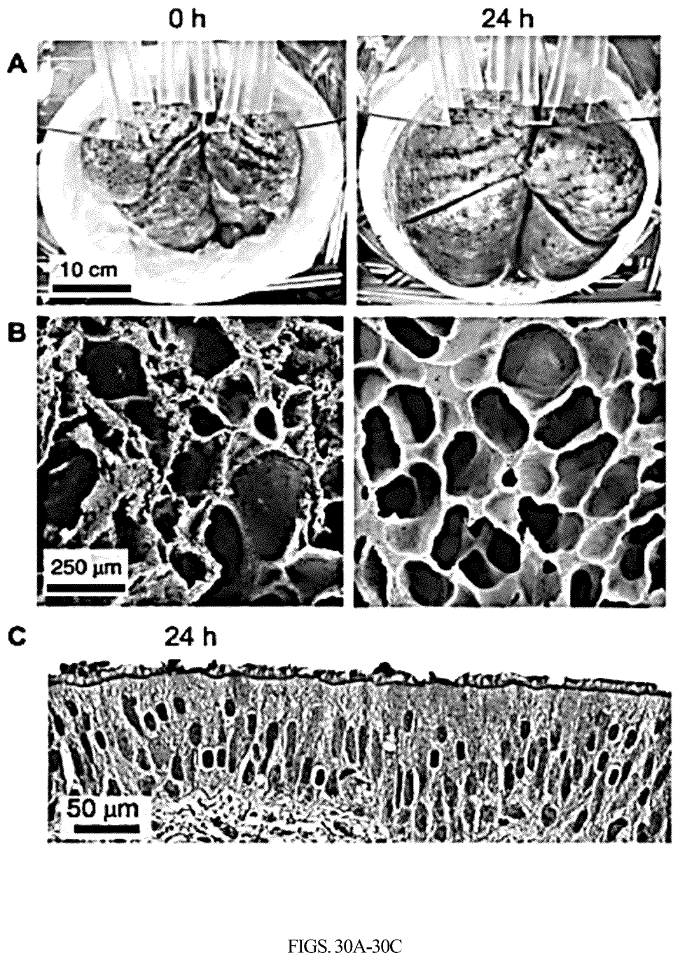

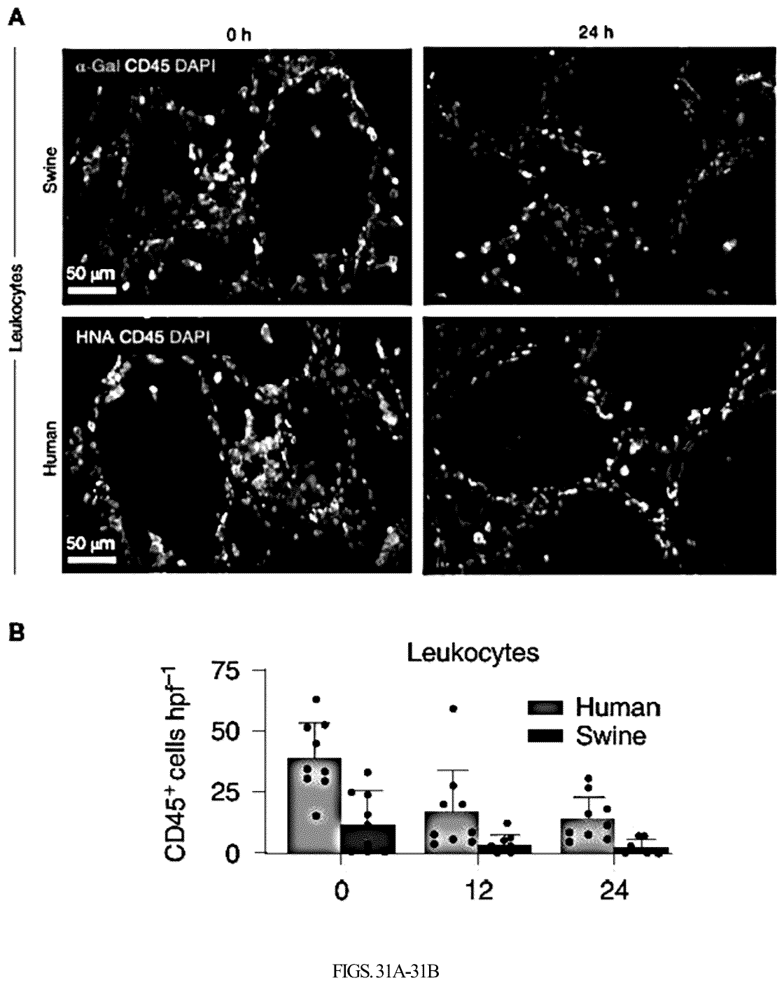

Preliminary Results: We support human lungs utilizing EVLP and a xenogeneic cross-circulation platform. Our group has demonstrated successful support and regeneration of severely injured whole human lungs on cross-circulation with a porcine host for 24 hours. Human lungs rejected for transplant due to various reasons such as aspiration pneumonitis and multi-lobar consolidation were placed on xenogeneic cross-circulation which resulted in improved lung morphology and function while maintaining vasculature and airway integrity (FIG. 30). With the addition of cobra venom factor to the immunosuppressive therapy, the risk of acute rejection was mitigated. Furthermore, human lung exposure to circulating swine xenoantigens and leukocytes was only transient, and no abnormal physiology or morphology was observed in the vasculature (FIG. 31). Notably, the porcine host supporting the human ex vivo lungs remained hemodynamically and biochemically stable throughout cross-circulation (35).

Generate CF-mimetic ex vivo human lung utilizing biologically accurate artificial CF mucus: Given the limited availability of human CF lungs, we will develop and validate a CF-mimetic ex-vivo whole human lung model using bioartificial mucus generated above while utilizing human donor lungs declined for transplant and consented for research. We will compare bioartificial mucus delivery resultant functional changes to the ex-vivo whole porcine lung model developed above.

Inclusion criteria for human lung selection: Human lungs declined for transplantation will be consented for research use by local organ procurement organizations, via our established collaborations with Tennessee Donor Services, LiveOnNY, and Lung Bioengineering, (United Therapeutics, Inc). We will select lungs unsuitable for clinical transplantation that have potentially reversible acute injuries (aspiration, contusion, infection) without evidence of sepsis, severe trauma, or chronic parenchymal disease (interstitial lung disease, COPD). Recorded donor characteristics will include age, sex, cause of death, underlying lung disease, smoking history, chest imaging, time on ventilator, time from brain death to procurement, PaO₂/FiO₂, BAL cultures, single vs double lung, donation status (brain death vs. cardiac death), and use of clinical EVLP. Baseline tissue specimens will be collected. The lung procurement will follow standard clinical practice. Unlike in syngeneic animal research, there is an inherent variability in the characteristics of human donor lungs used both for research and transplantation. Our published data show that despite organ variability, our xenogeneic XC platform consistently rehabilitates human lung function (35, 36).

Delivery, validation, and mucociliary clearance of bioartificial mucus in human lungs ex-vivo: CF-mimetic mucus will be introduced to human lungs on positive pressure ventilation in similar fashion as above. Given the differences in airway morphology of porcine and human airways, we will look for any key differences in mucus delivery, delivery verification, and clearance between the porcine and human lungs. We will utilize the experimental data in conjunction with mathematical modeling to assess the distributions of bioartificial mucus into the large and smaller airways. The bioartificial mucus delivery to the ventilated human lungs will be verified by bronchoscopy, ICG/NIR imaging, and fluorescent labeling with CFSE. Mucociliary clearance will be evaluated by placing human lungs on EVLP and by analyzing mucus samples (e.g., pH, modulus, composition) at different airway generations and timepoints. Dosing volumes and frequencies will be compared to the porcine equivalent.

Functional changes in human lungs after mucus delivery: Mechanical properties and functional changes of human lungs after bioartificial mucus delivery to ventilated ex-vivo human lungs will be evaluated by our non-destructive measurements of tissue elasticity and whole lung compliance. The measured values will be compared to the porcine data obtained to understand the differences between the lungs obtained from two different sources.

Investigate nanotherapeutic delivery methods in human CF-mimetic lungs on EVLP: We will investigate nanotherapeutic delivery and evaluate nanoparticle distribution in human lungs supported by EVLP. Outputs will be benchmarked to the porcine outputs to optimize and validate the porcine CF-mimetic model.

Nanoparticle delivery and real-time localization: Human lungs with bioartificial mucus will be supported using EVLP for up to 12 hours to evaluate nanoparticle delivery and localization. Mucus will be introduced via methods described above. As previously described, “empty” nanoparticles (without therapeutic cargo) will be delivered via aerosolization and bronchoscopy, and their distribution throughout the lung will be assessed by: (i) fluorescently labeled nanoparticles in conjunction with transpleural fluorescent microscopy, (ii) radiopaque metallic nanoparticles and the use of X-ray, and (iii) near-infrared imaging of nanoparticles fused with ICG. These methods will help determine the kinetics of distribution throughout the airways and allow for real-time localization of nanoparticles to elucidate the most effective nanoparticle delivery method and characterize nanoparticle localization in the CF-mimetic human lung. Effects of lung preconditioning by frequency oscillation, nebulized hypertonic saline, or dornase-alpha on nanoparticle delivery will also be investigated. The results of this sub-aim will be systematically compared to those utilizing the CF-mimetic porcine model.

Evaluate cellular uptake of nanotherapeutics in human CF-mimetic lungs on cross circulation: This will parallel porcine lung studies with studies of human lungs unsuitable for transplant. We will develop an ex-vivo CF-mimetic human lung model with bioartificial mucus and support the lungs on a xenogeneic platform for real-time evaluation of lung function and nanotherapeutic uptake and efficacy. Outputs will be benchmarked against the corresponding CF-mimetic porcine model for cross-validation.

Model system: Human lungs with bioartificial mucus will be supported on xenogeneic cross-circulation for up to 48 hours (or longer as needed) to deliver nanotherapeutics and monitor therapeutic efficacy in similar fashion as described and as previously described by our group (35). Bioartificial mucus will be introduced via methods described above. Cobra venom factor will be added to the immunosuppressive regimen to deplete the complement cascade and prevent acute rejection of human lungs supported with xenogeneic cross-circulation (35).

Monitoring cellular uptake and clearance of delivered nanoparticles: Fluorescently labeled nanotherapeutics with CFTR cargo will be delivered to the human CF-mimetic lungs by targeted bronchoscopy or nebulization. Functional lung parameters after therapeutic intervention will be monitored. Using fluorescent microscopy of periodic distal lung biopsies, we will evaluate retention and clearance of nanoparticles within the mucus, as described above. We will evaluate nanotherapeutic uptake utilizing a luciferase reporter gene and whole lung bioluminescent imaging.

CFTR expression in CF-mimetic human lungs following therapeutic delivery: Genetic cargo (e.g., CFTR, reporter genes) expression in targeted human lung regions will be investigated via methods described above. We will obtain lung biopsies/wedge resections at various timepoints and perform RNA sequencing, PCR, Western blot, and immunofluorescent staining to evaluate expression at both RNA and protein levels. The bioimpedance probe will also be used in the CF-mimetic human lung model. Given the innate CFTR expression in human lungs, we will measure CFTR expression in human lungs prior to introduction of nanoparticles to establish a baseline. Additionally, we will evaluate changes in mucus composition, mucus viscosity, lung clastic modulus, and lung functional parameters after gene delivery as previously described above.

Expected Outcomes: This is to validate nanotherapeutic delivery and efficacy in CF-mimetic human lungs and to benchmark the CF-mimetic porcine lung model against corresponding studies in human lungs. The proposed studies will validate: (i) optimal CF-mimetic mucus delivery to the human lung, (ii) nanotherapeutic delivery, and (iii) quantify therapeutic efficacy in the human lung to benchmark against the CF-mimetic porcine model. Materials and methods developed towards modeling CF ex vivo will be shared with the CF research community to enable therapeutic testing across different research areas and sites.

In addition to testing inhaled therapeutics in development, this CF model also has other practical applications including to study mucociliary clearance, pulmonary infection and bacterial biofilms, cellular tropism of gene therapy, and lung mechanics, among others.

### REFERENCES

Chance D L, Mawhinney T P. Observations of, and Insights into, Cystic Fibrosis Mucus Heterogeneity in the Pre-Modulator Era: Sputum Characteristics, DNA and Glycoprotein Content, and Solubilization Time. Journal of Respiration. 2021; 1 (1): 8-29.
Kirchner S, Fothergill J L, Wright E A, James C E, Mowat E, Winstanley C. Use of Artificial Sputum Medium to Test Antibiotic Efficacy Against Pseudomonas aeruginosa in Conditions More Relevant to the Cystic Fibrosis Lung. J Vis Exp. 2012 (64): 3857.
Mir S M, Chen J, Pinezich M R, O'Neill J D, Huang S X L, Vunjak-Novakovic G, et al. Imaging-guided bioreactor for de-epithelialization and long-term cultivation of ex vivo rat trachea. Lab on a Chip. 2022; 22 (5): 1018-31.
Mir S M, Chen J, Pinezich M R, O'Neill J D, Guenthart B A, Vunjak-Novakovic G, et al. Imaging-Guided Bioreactor for Generating Bioengineered Airway Tissue. Journal of visualized experiments: JOVE. 2022 (182).
Chen J, Mir S M, Pinezich M R, O'Neill J D, Guenthart B A, Bacchetta M, et al. Homogeneous Distribution of Exogenous Cells onto De-epithelialized Rat Trachea via Instillation of Cell-Loaded Hydrogel. ACS Biomaterials Science & Engineering. 2021; 8 (1): 82-8.
Kim J, Olsen T R, Zhu J, Hilton J P, Yang K, Pei R, et al. Integrated microfluidic isolation of aptamers using electrophoretic oligonucleotide manipulation. Scientific reports. 2016; 6 (1): 1-10.
Kim J, Hilton J P, Yang K-A, Pei R, Stojanovic M, Lin Q. Nucleic acid isolation and enrichment on a microchip. Sensors and Actuators A: Physical. 2013; 195:183-90.
Jiang K, Yan H, Rickert C, Marczynski M, Sixtensson K, Vilaplana F, et al. Modulating the Bioactivity of Mucin Hydrogels with Crosslinking Architecture. Advanced Functional Materials. 2021; 31 (10): 2008428.
Tan M, Mao Y, Walker T W. Rheological Enhancement of Artificial Sputum Medium. Applied Rheology. 2020; 30 (1): 27-38.
Tuon F F, Dantas L R, Suss P H, Tasca Ribeiro V S. Pathogenesis of the Pseudomonas aeruginosa Biofilm: A Review. Pathogens. 2022; 11 (3): 300.
Kim J, O'Neill J D, Vunjak-Novakovic G. Rapid retraction of microvolume aqueous plugs traveling in a wettable capillary. Applied physics letters. 2015; 107 (14): 144101.
Kim J, O'Neill J D, Dorrello N V, Bacchetta M, Vunjak-Novakovic G. Targeted delivery of liquid microvolumes into the lung. Proceedings of the National Academy of Sciences. 2015; 112 (37): 11530-5.
Hozain A E, Tipograf Y, Pinezich M R, Cunningham K M, Donocoff R, Queen D, et al. Multiday maintenance of extracorporeal lungs using cross-circulation with conscious swine. The Journal of Thoracic and Cardiovascular Surgery. 2020; 159 (4): 1640-53. e18.
Halpern D, Jensen O, Grotberg J. A theoretical study of surfactant and liquid delivery into the lung. Journal of Applied Physiology. 1998; 85 (1): 333-52.
Espinosa F, Shapiro A, Fredberg J, Kamm R. Spreading of exogenous surfactant in an airway. Journal of Applied Physiology. 1993; 75 (5): 2028-39.
Chen J, Mir S M, Pinezich M R, O'Neill J D, Guenthart B A, Bacchetta M, et al. Non-destructive vacuum-assisted measurement of lung elastic modulus. Acta Biomaterialia. 2021.
Bou Jawde S, Takahashi A, Bates J H, Suki B. An analytical model for estimating alveolar wall elastic moduli from lung tissue uniaxial stress-strain curves. Frontiers in physiology. 2020; 11:121.
Suki B, Bates J H. Lung tissue mechanics as an emergent phenomenon. Journal of applied physiology. 2011; 110 (4): 1111-8.
Hochmuth R M. Micropipette aspiration of living cells. Journal of biomechanics. 2000; 33 (1): 15-22.
Sakai A, Murayama Y, Yanagisawa M. Cyclic Micropipette Aspiration Reveals Viscoelastic Change of a Gelatin Microgel Prepared Inside a Lipid Droplet. Langmuir. 2020; 36 (19): 5186-91.
Sato M, Theret D, Wheeler L, Ohshima N, Nerem R. Application of the micropipette technique to the measurement of cultured porcine aortic endothelial cell viscoelastic properties. 1990.
Theret D, Levesque M, Sato M, Nerem R, Wheeler L. The application of a homogeneous half-space model in the analysis of endothelial cell micropipette measurements. 1988.
Kim J, Guenthart B, O'Neill J D, Dorrello N V, Bacchetta M, Vunjak-Novakovic G. Controlled delivery and minimally invasive imaging of stem cells in the lung. Scientific reports. 2017; 7 (1): 1-13.
Guenthart B A, O'Neill J D, Kim J, Queen D, Chicotka S, Fung K, et al. Regeneration of severely damaged lungs using an interventional cross-circulation platform. Nature communications. 2019; 10 (1): 1-16.
O'Neill J D, Guenthart B A, Kim J, Chicotka S, Queen D, Fung K, et al. Cross-circulation for extracorporeal support and recovery of the lung. Nature Biomedical Engineering. 2017; 1 (3): 1-15.
Johansson M E, Hansson G C. Preservation of mucus in histological sections, immunostaining of mucins in fixed tissue, and localization of bacteria with FISH. Mucins: Springer; 2012. p. 229-35.
Chen J, Mir S M, Pinezich M R, O'Neill J D, Guenthart B A, Bacchetta M, et al. Non-destructive vacuum-assisted measurement of lung elastic modulus. Acta Biomaterialia. 2021; 131: 370-80.
Guenthart B A, O'Neill J D, Bacchetta M. Cannulation Strategies in Ex Vivo Lung Perfusion. ASAIO Journal (American Society for Artificial Internal Organs: 1992). 2021.
Cypel M, Yeung J C, Hirayama S, Rubacha M, Fischer S, Anraku M, et al. Technique for prolonged normothermic ex vivo lung perfusion. The Journal of heart and lung transplantation. 2008; 27 (12): 1319-25.
Tang Y, Yan Z, Lin S, Huntemann E D, Feng Z, Park S-Y, et al. Repeat Dosing of AAV2. 5 T to Ferret Lungs Elicits an Antibody Response That Diminishes Transduction in an Age-Dependent Manner. Molecular Therapy-Methods & Clinical Development. 2020; 19: 186-200.
Cooney A L, Sinn P L. Intratracheal aerosolization of viral vectors to newborn pig airways. BioTechniques. 2020; 68 (5): 235-9.
Cypel M, Liu M, Rubacha M, Yeung J C, Hirayama S, Anraku M, et al. Functional repair of human donor lungs by IL-10 gene therapy. Science translational medicine. 2009; 1 (4): 4ra9-4ra9.
Mangal S, Gao W, Li T, Zhou Q T. Pulmonary delivery of nanoparticle chemotherapy for the treatment of lung cancers: challenges and opportunities. Acta pharmacologica sinica. 2017; 38 (6): 782-97.
Yang L, Gradl R, Dierolf M, Möller W, Kutschke D, Feuchtinger A, et al. Multimodal precision imaging of pulmonary nanoparticle delivery in mice: dynamics of application, spatial distribution, and dosimetry. Small. 2019; 15 (49): 1904112.
Hozain A E, O'Neill J D, Pinezich M R, Tipograf Y, Donocoff R, Cunningham K M, et al. Xenogeneic cross-circulation for extracorporeal recovery of injured human lungs. Nature Medicine. 2020; 26 (7): 1102-13.
Wu W K, O'Neill J D, Guenthart B A, Hozain A E, Stokes J W, Ukita R, Patel Y J, Pinezich M R, Talackine J R, Cardwell N L, Fung K, Vunjak-Novakovic G, Bacchetta M. Technique for xenogeneic cross-circulation to support human donor lungs ex vivo. The Journal of Heart and Lung Transplantation, epublished on Nov. 14, 2022.doi::https://doi.org/10.1016/j.healun.2022.11.002

1. A mucosal tissue bioreactor comprising:
a base comprising one or more wells that support a culture medium;
one or more platforms configured to support a portion of an airway mucosa in at least partial contact with the culture medium in the one or more wells;
a fluid circulation loop that circulates the culture medium through the one or more wells; and
an air circulation loop that circulates air over a top surface of the portion of the airway mucosa.

2. An air-liquid interface bioreactor with integrated perfusion and ventilation that supports a slice of mammalian airway mucosa in culture comprising:
a base comprising one or more wells for holding a culture medium;
one or more platforms for supporting the mucosa in contact with the culture medium in the one or more wells;
a fluid circulation loop for circulating culture medium through the one or more wells; and
an air circulation loop for circulating air over a top surface of the mucosa.

3. The bioreactor of claim 1, wherein the one or more wells are surrounded on a horizontal plane thereof by vertical walls so as to keep fluid in said wells within wells.

4. The bioreactor of claim 1, wherein each of the one or more wells comprises an entry side and a removal side separated by a half-wall that has a height smaller than a height of remaining walls of the well;
wherein the entry side is in fluid communication with inlet tubing of the fluid circulation loop; and
the removal side is in fluid communication with outlet tubing of the fluid circulation loop so that culture medium level is maintained at the height of the half-wall on the entry side.

5. The bioreactor of claim 1, wherein one or more platforms comprise a chair-like tissue holder comprising a polymeric frame with a permeable mesh seat on which the portion of the airway mucosa is positioned and one or more legs, the legs having a long axis substantially perpendicular to a plane of the permeable mesh seat.

6. The bioreactor of claim 5, wherein the portion of the airway mucosa is attached to the permeable seat portion of the chair-like tissue holder with a collagen hydrogel or other adhesive.

7. The bioreactor of claim 5, wherein the chair-like tissue holder comprises four legs, which plug into complementarily-shaped holes in a floor of the reactor so as to stabilize the holder's position.

8. The bioreactor of claim 5, wherein the permeable mesh seat of the chair-like tissue holder is disposed near the height of the culture medium in the one or more wells so that the portion of the airway mucosa positioned thereon is maintained in an air-liquid interface wherein a top surface of the portion of the airway mucosa is exposed to air and a bottom of the portion of the airway mucosa is submerged in the culture medium.

9. The bioreactor of claim 8, wherein an airflow is introduced to the top surface of the portion of the airway mucosa and a perfusate is introduced to the submerged bottom of the portion of the airway mucosa.

10. The bioreactor of claim 1, comprising:
a lid comprising a central opening above the location of the tissue in its chair-like tissue holder(s), wherein the opening is optionally:
(i) left open to the air for easy loading and removal of the entire mucosa airway slice and/or chair-like tissue holder;
(ii) sealed with a coverslip and adhesive;
(iii) sealed with an insert that holds a recessed coverslip deeper into the bioreactor than the remainder of the lid for the purpose of bringing imaging equipment in closer proximity to the surface of the tissue; and/or
(iv) sealed with an insert that comprises a top window comprising a transparent material and/or material compatible with visual or other microscopy.

11. The bioreactor of claim 10, wherein the lid is detachable, and is securable by one or more pins, wherein an airtight seal is formed with the remainder of the bioreactor when the lid is secured by one or more pins.

12. The bioreactor of claim 10, wherein the lid is securable by two pins, positioned on opposite outside ends of outside surfaces of walls of the bioreactor.

13. The bioreactor of claim 1, comprising one or more windows in a side wall of the reactor positioned such that a side profile of the portion of the airway mucosa, tissue holder, culture medium below the portion of the airway mucosa and mucus/air above the portion of the airway mucosa are observable.

14. The bioreactor of claim 1, further comprising a peristaltic pump and a heating system employing a heating pad with a PID controller and digital readout.

15. A plurality of bioreactors of claim 1, each comprising a portion of an airway mucosa slice.

16. The plurality of bioreactors of claim 15, wherein the plurality of bioreactors are maintained in parallel in a standard tissue incubator.

17. The bioreactor of claim 1, further comprising the portion of the airway mucosa supported on the platform.

18. The bioreactor of claim 17, further comprising bioartificial mucus on a top surface of the portion of the airway mucosa.

19. A system comprising a whole lung model of cystic fibrosis comprising:
(i) an ex vivo lung supported with a cross-circulation apparatus;
(ii) bioartificial mucus present in the ex vivo lung airways which mimics mucus of human cystic fibrosis lungs;
(iii) integrated real-time imaging and/or analytic tools for assessment of lung function and therapeutic uptake and efficacy.

20. The system of claim 19, wherein the cross-circulation apparatus comprises:
an extracorporeal organ chamber for containing and supporting the ex vivo lung, wherein the chamber comprises a time- and temperature-controlled humidifier and misting spray onto the ex vivo lung and into the chamber to maintain a humid environment within the chamber; and
an extracorporeal cross-circulation circuit for connecting ex vivo lung with a host organism for cross-circulation of blood or plasma, which circuit comprises:
fluid conduits providing flow of the blood or plasma between a vascular system of the host organism to vasculature of the ex vivo lung;
an arterial pressure sensor for sensing extracorporeal in-flow line pressure;
a venous pressure sensor for sensing extracorporeal out-flow line pressure; and
a circulating pump which regulates blood or plasma flow within a predetermined percentage range of cardiac output of the host organism based on a trans-organ pressure difference between arterial and venous pressure measured by the arterial pressure sensor and the venous pressure sensor;
determining a status of the lung before and after administration of the candidate therapeutic for cystic fibrosis so as to determine if the candidate therapeutic improves one or more symptoms of cystic fibrosis.
